(19) United States
(12) Patent Application Publication
(10) Pub. No.: US 2025/0222446 A1
(43) Pub. Date: Jul. 10, 2025

(54) SHAPED MOVTETAOX AND MOVTENBOX CATALYST WITH HIGH STRENGTH AND ODH PERFORMANCE

(71) Applicant: NOVA CHEMICALS (INTERNATIONAL) S.A., Fribourg (CH)

(72) Inventors: David SULLIVAN, Calgary (CA); Vasily SIMANZHENKOV, Calgary (CA); Hanna DRAG, Calgary (CA)

(21) Appl. No.: 18/852,967

(22) PCT Filed: Mar. 8, 2023
(86) PCT No.: PCT/IB2023/052209

§ 371 Date: Sep. 30, 2024

Related U.S. Application Data
(60) Provisional application No. 63/326,559, filed on Apr. 1, 2022.

(51) Int. Cl. B01J 37/00; B01J 21/08; B01J 23/00; B01J 27/057; B01J 35/31; B01J 35/37; B01J 35/40; B01J 35/54; B01J 37/04; B01J 37/08; C07C 5/32

(52) U.S. Cl. CPC B01J 37/0063; B01J 21/08; B01J 23/002; B01J 27/0576; B01J 35/31; B01J 35/37; B01J 35/40; B01J 35/54; B01J 37/0018; B01J 37/04; B01J 37/08; C07C 5/324; B01J 2523/55; B01J 2523/56; B01J 2523/64; B01J 2523/68

(57) ABSTRACT

Shaped catalyst compositions and methods for making and using the shaped catalyst compositions are provided. In an exemplary a catalyst active phase includes a MoVTeNbOx catalyst. The composition also includes a support phase, wherein the support phase includes fumed silica, and wherein the catalyst active phase and support phase form a heterogeneous mixture.

### TECHNICAL FIELD

This document relates to heterogeneous shaped catalysts, methods of synthesizing the catalysts, and methods of using the catalysts.

### BACKGROUND ART

Olefins like ethylene, propylene, and butylene, are basic building blocks for a variety of commercially valuable polymers. Since naturally occurring sources of olefins do not exist in commercial quantities, polymer producers rely on methods for converting the more abundant lower alkanes into olefins. The method of choice for today's commercial scale producers is steam cracking, a highly endothermic process where steam-diluted alkanes are subjected very briefly to a temperature of at least 800° C. The fuel demand to produce the required temperatures and the need for equipment that can withstand that temperature add significantly to the overall cost. In addition, the high temperature promotes the formation of coke, which accumulates within the system, resulting in the need for costly periodic reactor shutdown for maintenance and coke removal.

Selective oxidation processes, such as oxidative dehydrogenation (ODH), are an alternative to steam cracking that are exothermic and produce little or no coke. In ODH, a lower alkane, such as ethane, is mixed with oxygen in the presence of a catalyst and optionally an inert diluent, such as carbon dioxide, methane, nitrogen or steam, at temperatures as low as 300° C. to produce the corresponding alkene. Various other oxidation products may be produced in this process, including carbon dioxide and acetic acid, among others. ODH suffers from lower conversion rates when compared to steam cracking, a fact that when combined with lower selectivity and the risk of thermal explosion due to mixing of a hydrocarbon with oxygen, may have prevented ODH from achieving widespread commercial implementation.

Shaped catalysts can be used in ODH of ethane. Shaping is important when creating a heterogeneous catalyst. For a fixed bed catalyst, the shape of the catalyst material facilitates many critical things, including pressure drop across the reactor, heat and mass transfer, the avoidance of fines during loading and handling, catalyst attribution/breakdown during process operations, and catalyst costs.

### SUMMARY OF INVENTION

This disclosure describes a fumed silica formulation that produces a shaped catalyst with very high mechanical strength, while maintaining excellent performance, as measured by activity and ethylene selectivity, for the oxidative dehydrogenation (ODH) of ethane.

In an implementation, a composition is provided that includes a catalyst active phase, wherein the catalyst active phase includes a MoVTeNbOx catalyst. The composition also includes a support phase, wherein the support phase includes fumed silica, and wherein the catalyst active phase and support phase form a heterogeneous mixture.

In an aspect, the composition includes a binder, wherein the binder includes a polymer organic acid, amine, or alcohol. In an aspect, the binder includes polyvinyl alcohol or polyethylene glycol, or both. In an aspect, the binder includes 60-80 wt. % polyvinyl alcohol and 20-40 wt. % polyethylene glycol.

In an aspect, the composition further includes an inorganic binder, wherein the inorganic binder includes talc or calcium carbonate.

In an aspect, the composition is sintered to form a ceramic.

In an aspect, the composition further includes a plasticizer, wherein the plasticizer includes polyethylene glycol.

In an aspect, the composition further includes a lubricant. In an aspect, the lubricant is 1-2 wt. % of the composition, and the lubricant includes graphite.

In an aspect, the fumed silica is 5-95 wt. % of the composition.

In an aspect, the composition is formed into a pressed tablet, wherein the pressed tablet has a density of 1.0-1.5 g/cm³.

In an aspect, the composition is formed into a sintered ceramic catalyst.

Another implantation described herein provides a method of making a shaped catalyst. The method includes combining a catalyst active phase, a support phase, and a binder in an aqueous solvent to form an aqueous solution, wherein the catalyst active phase includes MoVTeNbOx and the support phase includes fumed silica. The method also includes heating the aqueous solution to form a thick paste, drying the thick paste to evaporate the aqueous solvent to form a solid powder, grinding the solid powder to form granules, and pressing the granules to form a tablet.

In an aspect, grinding the solid powder to form granules includes grinding the solid powder to form granules wherein the granules are between 105 μm and 710 μm in diameter.

In an aspect, the method further includes debinding the tablet, wherein debinding the tablet includes heating the tablet to remove the binder, and sintering the tablet, wherein sintering the tablet includes heating the tablet to coalesce the tablet into a densified solid. In an aspect, debinding the tablet includes heating the tablet to 200° C.-400° C. at a rate of 1-2° C. per minute. In an aspect, sintering the tablet includes heating the tablet to 400° C.-600° C. at a rate of up to 5° C. per minute.

Another implementation described herein provides a method of making a shaped catalyst. The method includes combining a catalyst active phase, a support phase, and a binder in an aqueous solvent to form an aqueous solution, wherein the catalyst active phase includes MoVTeNbOx and the support phase includes fumed silica, heating the aqueous solution to form a thick paste, drying the thick paste to evaporate the aqueous solvent to form a solid powder. The solid powder is ground to form granules and the granules are pressed to form a tablet. The method includes debinding the tablet, wherein debinding the tablet includes heating the tablet to remove the binder, and sintering the tablet, wherein sintering the tablet includes heating the tablet to coalesce the tablet into a densified solid.

Another implementation described herein provides a method of oxidative dehydrogenation of ethane. The method includes flowing a feed gas through a microreactor unit, wherein the feed gas includes ethane, oxygen, and nitrogen, pressuring the feed gas in the microreactor, and contacting the feed gas with a shaped catalyst. The shaped catalyst includes a catalyst active phase, wherein the catalyst active phase includes a MoVTeNbOx catalyst, and a support phase, wherein the support phase includes fumed silica, wherein the catalyst active phase and support phase form a heterogeneous mixture. The method also includes heating the feed gas to 450° C.-470° C. and recovering ethylene gas.

In an aspect, the feed gas includes a 1:0.5 mole ratio of ethane to oxygen.

The details of one or more implementations of the disclosure are set forth in the accompanying drawings and the description that follows. Other features, objects, and advantages of the disclosure will be apparent from the description and drawings, and from the claims.

### BRIEF DESCRIPTION OF THE FIGURES

FIG. 1 shows an example schematic of a shaped catalyst.

FIG. 2 shows an example method of forming a shaped catalyst.

FIG. 3 shows a flow diagram of an example process of debinding and sintering a catalyst.

FIG. 4 is a flowchart of an example method of the oxidative dehydrogenation of ethane.

FIG. 5 shows the tabletability profile of the Shaped Catalyst 1.5 green body tablets (Shaped Catalyst 1.5 GB).

FIG. 6 shows the tabletability profile of the Shaped Catalyst 1.7 green body tablets (Shaped Catalyst 1.7 GB).

FIG. 7 shows the tabletability profile of the Shaped Catalyst 2.1 GB comparative example tablets and Shaped Catalyst 2.2 GB comparative example tablets.

FIG. 8 shows MRU data of the long-term ethane to ethylene conversion for the MoVTeTaOx catalyst-only Shaped Catalyst 1.2SB tablets.

FIG. 9 shows the long-term ethylene selectivity testing results of Shaped Catalyst 1.2SB tablets.

FIG. 10 shows a conversion MRU data plot for MoVTeTaOx supported Shaped Catalyst 1.3SB tablets.

FIG. 11 shows a selectivity MRU data plot for MoVTeTaOx supported Shaped Catalyst 1.3SB tablets.

FIG. 12 shows a conversion MRU data plot for MoVTeTaOx supported Shaped Catalyst 1.5SB tablets.

FIG. 13 show a selectivity MRU data plot for MoVTeTaOx supported Shaped Catalyst 1.5SB tablets.

FIG. 14 shows the long-term ethane conversion testing results of Shaped Catalyst 1.7SB tablets.

FIG. 15 shows the long-term ethylene selectivity testing results of Shaped Catalyst 1.7SB tablets.

FIG. 16 shows the long-term ethane conversion of the Shaped Catalysts 1.8SB tablets.

FIG. 17 shows the ethylene selectivity of the Shaped Catalyst 1.8SB tablets.

FIG. 18 shows the long-term ethylene selectivity testing of Shaped Catalyst 1.7SB and Shaped Catalyst 1.8SB, overlaid into a single plot.

Like reference symbols in the various drawings indicate like elements.

### DESCRIPTION OF EMBODIMENTS

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Provided in this disclosure, in part, are compositions and methods for the conversion of ethane to ethylene. The compositions include a shaped catalyst, i.e., a mixture of heterogeneous catalyst components that can be compacted into a predefined shape, for example, a cylinder. The shaped catalysts include a catalyst active phase, support, and binder. The catalyst active phase is the component responsible for catalyzing the conversion of ethane, in the presence of oxygen, to ethylene. In some implementations, the catalyst active phase includes an MoVTeTaOx catalyst. The MoVTeTaOx catalyst includes molybdenum (Mo), vanadium (V), tellurium (Te), tantalum (Ta), and oxygen (O). The catalyst is represented by the formula MoVTeTaOx. In some implementations, the ratio of elements in the catalyst active phase is Mo₁V_{0.66}Te_{0.17}Ta_{0.09}Ox. In some implementations, the ratio of elements in the catalyst active phase is Mo₁V_{0.31}Te_{0.22} Ta_{0.10}Ox. In some implementations, the catalyst active phase includes an MoVTeNbOx catalyst.

The support in a shaped catalyst is important for the properties of the resulting shaped catalyst. For example, the support facilitates shaping and mechanical strength of the support catalyst, can avoid fines during loading, can prevent catalyst attrition during a process operation, and can also be one of the ways to reduce the overall cost of the shaped catalyst. The shape and support of the catalyst also improves heat and mass transfer, can provide pores and channels to permit gas products in and out of the shaped catalyst, and can facilitate heat transfer away from the catalyst and the reactants. Further, the shape and size of the shaped catalyst can be responsible for pressure drop design across the reactor. The support can also reduce the overall cost of manufacturing the shaped catalyst by dispersing catalyst particle through the support, allowing reactants easier access to active sites. Accordingly, the support plays a number of roles in a shaped catalyst. Examples of support/catalyst carriers include precipitated silica, fumed silica, and alpha-alumina. The shaped catalysts described herein are synthesized with fumed silica.

The shaped catalysts herein includes a fumed silica formulation that supports the catalyst and produces a shaped catalyst with very high mechanical strength, while maintaining excellent performance as shown by activity and ethylene selectivity for the oxidative dehydrogenation of ethane.

Adding support to the catalyst tablets improved overall catalyst performance compared to a catalyst-only tablet. This was a surprising result. Without wishing to be bound by theory, the improved performance of the shaped catalysts may be due to the improved ability of feedstock gases to access all of the catalyst particles in the shaped catalyst tablets, despite the dense compaction of the shaped catalyst. In theory, increasingly dense catalyst tablets associated with high mechanical strength arises due to a lack of porosity. However, by including silica support in the catalysts, the catalysts retain some porosity without compromising mechanical strength. In addition to high mechanical strength, the shaped catalysts that include fumed silica display new material properties as the result of the inclusion of a fumed silica support. Typically, silica is generally a challenging material to enlarge (agglomerate) into mechanically strong macroscopic shapes. For example, it is difficult to agglomerate silica into shapes larger than 200 μm, which is typical for a spray-dried silica applied in other heterogeneous technologies like gas phase polyethylene production. However, as described herein, catalysts that include fumed silica with an oxidative dehydrogenation catalyst active phase result in a catalyst material that has unique properties, different from the fumed silica alone. The shaped catalyst with fumed silica support material has processability properties that surpass other tablet pressing ceramic materials, such as alumina. This property is exemplified by the tabletability profiles, i.e., a comparison of the radial tensile strength of a tablet as a function of compaction pressure. Tabletability profiles are discussed herein in Examples 2 and 3. Further, the catalyst tablets described herein have remarkably high density and mechanical strength, as shown in Examples 5 and 6.

The shaped catalysts described herein also include a binder. The binder in the shaped catalysts described herein is an additive that aids in agglomeration and makes the overall mixture more formable. In some implementations, organic additives can be used as a binder. Suitable organic binders include polymer organic acids, amines, or alcohols that can allow cross-linking or particles, for example polyvinyl alcohol. In some implementations, inorganic binders can be used. Suitable inorganic binders include soft (low Mohs hardness value) salt minerals, for example talc or calcium carbonate. In some implementations, the binder can be a mixture of two or more binders. For example, the binder can include a mixture of polyvinyl alcohol (PVA) and polyethylene glycol (PEG). In some implementations, the binder includes 80 wt. % PVA and 20 wt. % PEG. In some implementations, the binder includes 60 wt. % PVA and 40 wt. % PEG.

In some implementations, the shaped catalysts can also include a plasticizer, lubricant, or both a plasticizer and lubricant. A plasticizer is an additive that acts on an organic binder. The combination of an organic binder and a plasticizer is more plastic than the two ingredients separately. The amount and type of plasticizer can be chosen based on the ability of the plasticizer to lower the glass transition temperature of a polymeric binder. In some implementations, the plasticizer can include polyethylene glycol.

In some implementations, the shaped catalyst also includes an intergranular or intragranular lubricant. The lubricant is an additive added to granulated powder to help compressed shapes release more easily from a die cavity, and can prevent equipment jamming and excess wear.

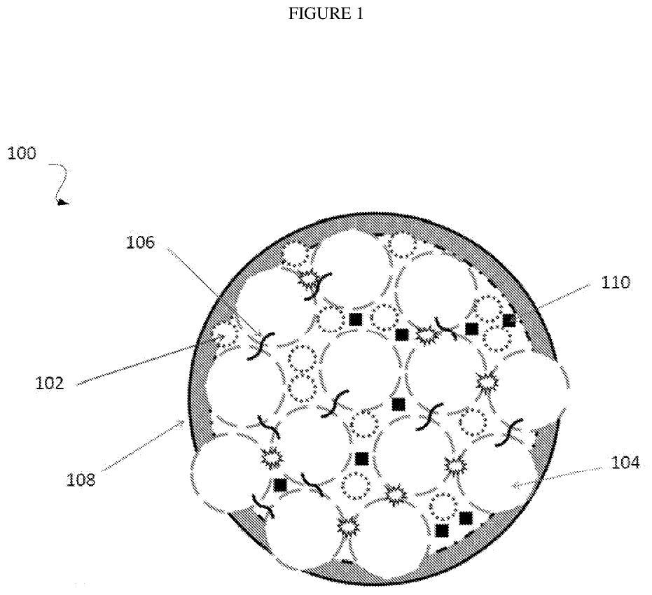

FIG. 1 shows an example schematic of a Shaped Catalyst 100. The Shaped Catalyst 100 can include active phase catalyst particles 102, support particles 104, binder 106, intergranular lubricant 108, and intragranular lubricant 110. A Shaped Catalyst 100 can include any combination of features 102-110 but does not necessarily require all features 102-110. For example, the Shaped Catalyst 100 can include active phase catalyst particles 102, support particles 104, and binder 106.

The shaped catalysts described herein can be used to catalyze the oxidative dehydrogenation of ethane. The oxidative dehydrogenation of ethane is shown in Equation 1.

$${CH}_{3} {CH}_{3} + \frac{1}{2} {O}_{2} \to {CH}_{2} {C H}_{2} + {H}_{2} O \qquad Eq . 1$$

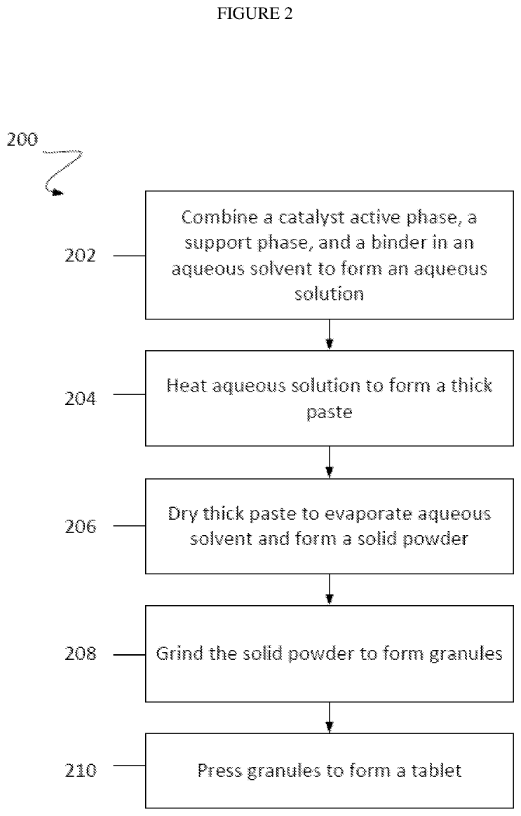

In some implementations, the shaped catalysts described herein include a MoVTeTaOx active catalyst phase. In some implementations, shaped catalysts described herein include a MoVTeNbOx active catalyst phase. Comparative examples of shaped catalysts with no active catalyst phase, or with alternative support phases, were also produced. The formulations of the shaped catalysts analyzed in Examples 1-11 are shown in Table 1. The silica support in Table 1 is fumed silica. However, in some embodiments, the silica can be other forms of silica. For example, precipitation silica (hydrated), fumed silica (small size dehydrated), or colloidal silica (small size hydrated), or any mixture thereof, can be used as a silica support. The silica support can be 5-95 wt. % of the composition. In some implementations, the silica is 40-80 wt. % of the composition. In some implementations, the tablets include intragranular lubricant. Lubricant was applied via dry mixing through hand tumbling in a drum for 5 mins. The shaped catalysts described herein and in Table 1 were synthesized using wet formulation, briquetting, granulation, and tablet pressing, as described in more detail herein. FIG. 2 shows an example method 200 of forming a shaped catalyst. At 202, a catalyst active phase, a support phase, and a binder are combined in an aqueous solvent to form an aqueous solution. At 204, the aqueous solution is heated to form a thick paste. At 206, the thick paste is dried to evaporate the aqueous solvent and form a solid powder. At 208, the solid powder is ground to form granules. At 210, the granules are pressed to form a tablet. In some implementations, the method further includes debinding and sintering the tablet. The methods of forming the shaped catalysts are described in more detail herein.

### Wet Formulation

Synthesizing the shaped catalysts includes wet formulation. The ingredients added to a formulation to produce a formable composition depend on both the specific shaping process to be used (for example, tablet press or extrusion), as well as what properties are being built into the final formed shape. In the catalyst formulation selection stage, the shape, processing equipment and composition all must be taken into consideration. Judicious choice of ingredients (for example, binders or lubricants) will impact how wet or dry powders agglomerate, process through equipment, compress under force, sinter, and resist abrasion/crushing forces. In addition, the composition and shape have a decisive impact on the ultimate performance of the catalyst. In addition to meeting the demand of the intended process unit, the shape and composition must also impart sufficient mechanical properties to survive transport, reactor loading, operation, and reactor unloading.

In order for the materials to be converted into a formable composition, the ingredients are blended into a uniform distribution to produce a homogenous mixture of catalyst components. Ingredients of the shaped catalysts described herein were wet formulated into homogenous pastes that were then dried and worked into granules. The wet-formulation process mixes the ingredients and starts the agglomeration process. The shaped catalysts described here were wet-formulated with bench top mixing equipment, for example beakers, oil baths, and low-shear overhead stirrers. Wet-formulation was performed under heat and mixing to evaporate excess water and transform the suspension of ingredients into a homogenous paste. The paste was then dried in an oven at 105-110° C. overnight.

### Briquetting/Agglomeration

After drying overnight, the resulting dry “cake” was then ground with a mortar and pestle and fed into a 40 mm diameter die set to form briquettes in a press. The 40 mm die set was loaded with 7 g of formulation powder, pressed under 12 metric tons of force, and held under this force for 60 seconds. The force applied to the formulation powder is a function of cross-sectional area, accordingly sufficient force should be used to result in an agglomeration of powder depending on the size of the briquette. Briquettes can be formed in a press. Alternatively, briquetting and granulation can be accomplished with a high shear mixer. The produced disc-shaped briquettes were extracted. Granules were produced from the briquettes by grinding the briquettes into particles and sieving the particles to collect the desired particle size distribution.

### Granulation

An important step in tablet pressing is to transform the fine powders of the individual components of the shaped catalyst into agglomerated, size-enlarged particles. This step is referred to as granulation. Granulation is important for several reasons.

Granulation can improve the flowability characteristics of materials so that they can flow from a hopper into a die cavity. The flowability of ceramic powders can be defined and measured using the definitions and methods available in ASTM D6103/D6103M. Powders with too small of a particle size will lead to ‘clumping,’ where particles do not flow past each other smoothly. The clumping of small particles can arise due to static electricity or increase Van der Waals interactions due to increased surface area relative to volume. These forces can overcome gravitational forces working on the particles. In addition, granulated particles compact better than an unmodified, fine powder, resulting in tablets with a higher and more consistent density. Without wishing to be bound by theory, granules display better compaction than fine powders because granules are capable of plastic deformation and/or fracture during the rearrangement of granulated particles when they are uniaxially compressed. Plastic deformation and/or fracture allows granules to fill void spaces and have more intergranular contacts with other particles.

It was discovered that granule particle size distribution affects mechanical strength, particularly for tablets manufactured using an autoloading mode of an autopress tablet equipment. Therefore, a study was undertaken to determine how different granule particle size distributions would affect autoloaded tablet crush strengths.

### Pressing

As described herein, the shaped catalysts are formed using tablet pressing, also referred to as uniaxial die pressing or dry pressing.

The tablets formulated as described in Table 1 were produced using a CPR-6 Automated press from DOTT. BONAPACE & C. SRL, of Milan, Italy or a CARVER R manual press from Carver, Inc. of Wabash, Indiana, USA. The CPR-6 autopress has an adjustable force up to 2.5 metric tons and exchangeable diameter die sets. The die set used herein was an 0.125 inch (3.175 mm) OD cylindrical cavity with flat-face punches. The tablets were formed by the combined pressing action of two punches in a die. In the first step of the press operation, the bottom punch is lowered in the die creating a cavity into which the catalyst material powder is fed, via a hopper show. The exact depth of the lower punch is precisely set to meter the volume of powder that fills the cavity. The excess is scraped from the top of the die by the hopper show. Next, the upper punch is brought down into contact with the powder and the force of the compression is delivered by high-pressure compression roll, which compresses the catalyst material together into a hard pellet in the shape of the ide. After the compression step, the lower punch is raised to eject the pellet from the die cavity. Lastly, the tablet is bumped off the tablet pressing stage by the hopper shoe during the subsequent die cavity filling. The pellet is knocked off the stage into a collection pan.

The compression force can be adjusted by moving the upper die up or down. The upper die set has a built in setting 1 to 8, where 8 is the highest compressive force. The compressive forces ranged from 520 MPa to 1040 MPa. In addition, the lower die adjustment can also change the resulting linear displacement and impact the final compressive force. The lower die can be screwed up or down to make the final tablet height either shorter (more compression) or taller (less compression). The lower die can be labeled with three settings “1”, “2”, and “3”. The lower die setting 3 is the highest compressive force and 1 is the lowest. Since compression is a function of the formulation of the tablet itself, the upper and lower die can be adjusted on a formulation to formulation basis.

The speed of the autopresser can also be adjusted. Lower speeds allow the compressed material to shape and conform to the die set, resulting in tablets that are stronger and less prone to “capping”, when the upper and lower part of the tablet separates horizontally from the main body. However, the speed of the autopressor should be high enough to provide sufficient throughput. It was found that speed setting “2” produces tablets with high crush strengths for a range of different formulation materials. The speed can be adjusted depending on the equipment used to produce the tablets.

In some implementations, the tablets can be formed using a CARVER press, a manual press with a force readout gauge that can form tablets under different compressive forces. The CARVER press can be used to form 6 mm diameter tablets.

Pressing the formulated granules results in green body tablets, i.e., tablets that have not been debinded or sintered. The green body tablets were analyzed to yield tabletability profiles. Tabletability profiles can be used to compare granule formulations' responses to compaction, where an ideal material will have increasing tensile strength with increasing compaction pressure. Tensile strengths of ideal formulations increase with compaction pressure until a maximum tensile strength is reached, after which additional compaction pressure does not improve mechanical strengths.

Tabletability profiles can also be used to interpolate the compaction pressure required to achieve a green body tablet with sufficient strength, i.e., at least 1-2 MPa of tensile strength. As described herein, a green body tablet strength must be high enough that the tablet can be ejected intact from the tablet press and sustain subsequent handling before debinding and sintering steps. The axial and radial crush strengths of the shaped catalysts and comparative examples are described in Examples 2-5. The tensile strengths used in tabletability profiles are typically calculated based on radial crush strengths.

Crush strengths (breaking forces, in Newtons) are often converted into tensile strengths (pressure, MPa) when comparing tablets of different dimensions, since tensile strength calculation takes into account tablet dimensions. Radial (or diametral) tensile strength is calculated based on the formula:

$${σ}_{c} = \frac{2 P}{π Dt} \qquad Eq . 2$$

where σₜ is the tensile strength in MPa, P is the breaking load (radial crush strength) in Newtons (N), D is the tablet diameter in millimeters, and t is the tablet height in millimeters. Radial tense strengths are typically used for generating tabletability profiles as well as tablet compaction and compression profiles.

### Debinding

As described herein, a green body tablet has been pressed but has not been debinded or sintered. Before the shaped catalyst can be utilized in catalytic reactions, for example, oxidative dehydrogenation, the shaped catalysts are debinded and sintered.

A debinded body table has undergone a debinding step to remove organic binders. This step is performed before moving to the final sintering step. In some implementations, the debinding step includes a heat treatment step. The heat treatment is carried out slowly during thermal debinding. The green body tablets are heated under air to combust off organic additives. During the debinding step, combustion produces water and carbon dioxide gases. Accordingly, the debinding step is carefully controlled to prevent high-pressure gas build up in the tables.

Debinding removes organic binders and plasticizers in the green body tablet. Therefore, the maximum temperature of the debinding step is based on the organic binders and/or plasticizers in the green body tablet. For example, pure polyvinyl alcohol (PVA) has a range of decomposition of about 200 to about 300° C., depending on the molecular weight distribution of the PVA. In another example, pure polyethylene glycol has a range of decomposition of about 150 to about 250° C., depending on the molecular weight distribution of the PEG. Therefore, if PEG is used as the binder in a shaped catalyst formulation, the minimum debinding temperature should be about 250° C.

In some implementations, the heat treatment occurs under an air atmosphere to promote combustion of the binder, for example polyvinyl alcohol. In some implementations, the ramp rate of the heat treatment step is 1-2° C./min or less to avoid cracking or rupturing the tablets due to carbon dioxide, steam, and other gases released during the heat treatment step. The maximum debinding temperature applied in the debinding processes described herein is 550-600° C. However, debinding temperatures herein are below 400° C. to avoid oxidizing the catalyst. For the MoVTeTaOx shaped catalysts described in Table 1 (Shaped Catalysts 1.2-1.7), debinding temperatures were 400° C. with a ramp rate of 1° C./min. Debinding temperatures did not exceed 400° C., as such elevated temperatures in the presence of an air atmosphere can lead to catalyst deactivation. Debinding was performed in a catalyst calcining furnace.

### Sintering

After debinding, the tablets undergo a sintering step to increase their mechanical strength and yield a ceramic final product. A ceramic is any of the various hard, brittle, heat-resistant and corrosion-resistant materials made by shaping and then firing an inorganic, nonmetallic material, such as clay, at a high temperature. The sintering step also creates bridges/bonds between particles and coalesces the particles into a densified solid with increased mechanical strength. Sintering is a kinetic phenomenon between particles and can happen naturally if sufficient time is allowed. However, high temperatures can greatly accelerate sintering, reducing sintering time to a matter of hours. For tablet described herein, heat treatment is carried out under inert atmosphere during thermal sintering to avoid catalyst oxidation. Sintering is typically done at a higher temperature than debinding, and since no additional, or fewer, gaseous products are released from the compacted tablets at this stage, ramping rates can be higher, for example up to 5° C./min. In some implementations, the maximum temperature applied during the sintering step is 600° C. The sintering step yields a final ceramic shaped catalyst that does not contain binders. Sintering can control both densification and grain growth. Densification reduces porosity in a sample, thereby making it denser and stronger. Grain growth is the process of grain boundary motion to increase the average grain size. Mechanical strength properties benefit from both a high relative density and a small grain size. Therefore, using higher ramp rates to prevent large grain size is desirable when sintering tablets. Although the losses of gaseous products during sintering steps are lower, some water in the form of steam can still escape from the shaped catalyst. The amount of water lost can be determined via the loss on ignition test (LOI), which is an ASTM test described in UOP954-11.

For the MoVTeTaOx catalysts described in Table 1, the sintering temperature applied was 600° C. with a ramp rate of 1.6° C./min. While the MoVTeTaOx shaped catalysts described in Table 1 were sintered at a ramp rate of 1.6° C./min, faster ramp rates can also be suitable. However, faster ramp rates could lead to weaker final materials, arising from thermal stresses inside each particle or sudden release of water, which could crack particles or reduced their mechanical integrity. The MoVTeTaOx and MoVTeNbOx catalysts in Table 1 were sintered in a catalyst calcining furnace. Sintering was done under nitrogen flow because, as described herein, temperatures above 400° C. in air can lead to catalyst deactivation.

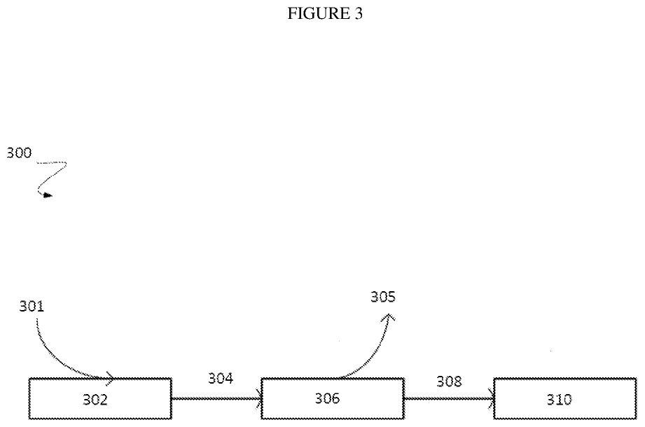

FIG. 3 shows a flow diagram of an example process 300 of debinding and sintering a catalyst. The green body tablet 302, including organic binders 301, is debinded at step 304 at 400° C. in air. Carbon dioxide (CO₂) gas 305 is released from the green body to yield a debinded body 306. Next, the debinded body is sintered at step 308 at 600° C., under an inert gas. In some implementations, the inert gas is nitrogen gas. Sintering yields a shaped catalyst 310 with increased mechanical strength. Surprisingly, the shaped catalysts described herein show high mechanical strength despite the lack of binders.

The shaped catalysts described herein were tested for their ability to catalyze the oxidative dehydrogenation of ethane using a microreactor unit (MRU). The MRU has a reactor tube made from stainless-steel SWAGELOK® Tubing, which had an outer diameter of 0.5 inches (1.27 cm), an internal diameter of about 0.4 inches (1.02 cm), and a length of about 13.4-15 inches (34.0-38.1 cm). Experimental temperatures of the MRU are measured using a 6-point WIKA Instruments Ltd. K-type thermocouple, which had an outer diameter of 0.125 inches (0.318 cm) and was inserted through the reactor. The 6-point thermocouple is used to measure and control the temperature within the catalyst bed. A room temperature stainless steel condenser is located after the reactor to collect water/acetic acid condensates. The gas product flow was allowed to either vent or was directed to an Agilent 8890 “hot gas” Gas Chromatograph (HGGC) during times when product gas analysis was required. Due to size restrictions during MRU testing, the catalyst pellets were gently crushed using a mortar and pestle into granules. The resulting granules are sieved and a particle size distribution between 425 μm and 1000 μm was collected to be loaded for testing on the MRU. In some implementations, the shaped tablets can be used as catalysts without granulation. Two loading methods were employed depending on whether or not the shaped catalyst contained a support, in order to maintain constant weight hourly space velocity (WHSV) of 5.46 h⁻¹.

For shaped catalysts with no support (i.e., Shaped Catalysts 1.2), 2.00 g of sieved catalyst from crushed pressed catalyst, with particle size ranging from 425 μm and 1000 μm, was physically mixed with quartz sand (50-70 mesh) such that the mixture produced a catalyst bed total volume of 6 mL. Once the catalyst bed was loaded into the reactor and connected to the MRU equipment, the testing was conducted as described herein.

For shaped catalysts with support, 4.00-5.00 g of pressed catalyst material, with particle size ranging from 425 μm and 1000 μm, was physically mixed with quartz sand such that the mixture produced a catalyst bed total volume of 6 mL. Since all catalyst materials were prepared with about 40 wt. % catalyst active phase, the weight loading of catalyst in the MRU testing equipment was in the range of 1.60-2.00 g.

For both supported and unsupported catalysts, the catalyst bed was loaded in the middle zone of the reactor—located between points 2 and 5 of the thermocouple—and the remaining volume of the reactor was packed with quartz sand. The reactor loading was then secured with glass wool on both the top and the bottom of the reactor. The quartz sand added to produce the catalyst bed volume of 6 mL was done to ensure the catalyst volume was sufficient to cover the thermocouple area between points 2 and 5. When catalysts were supported, the support increased the bed volume so that less sand was required to create a 6 mL bed volume.

For the MRU testing, a pre-mixed feed gas composition was achieved via upstream gas blending equipment involving calibrated mass flow controllers. The pre-mixed feed gas composition was fed through the reactor. The pre-mixed feed gas entering the reactor was 20 mol. % ethane, 10 mol. % oxygen, and 70 mol. % nitrogen (ethane-to-oxygen mole ratio of 1/0.5) and the reactor outlet was pressurized to 20 psig (0.14 MPa) using a back-pressure regulator. Furthermore, the pre-mixed feed gas flow was adjusted by a calibrated mass flow controller to obtain a constant weight hourly space velocity (WHSV) based on the weight of catalyst active phase in the overall catalyst bed.

The flow of the pre-mixed feed gas was controlled between 152-157 standard cubic cm (sccm) depending on weight loading to achieve a constant WHSV of 5.46 h⁻¹. The gas hourly space velocity (GHSV) was calculated based off the measured volume of the pressed particles (before mixing with quartz sand) and varied depending on each catalyst or catalyst material bulk density. Where WHSV is defined as mass flow of feed gas to the reactor divided by the weight of the catalyst in the catalyst bed, GHSV is defined as volumetric flow of the reactor feed gas divided by the volume of the catalyst bed. For GHSV values of supported catalysts, the catalyst bed includes diluent (including support volume). GHSV values for catalyst only could be calculated, where the total volume of the catalyst material measured when multiplied by the wt. % of catalyst is calculated to be the volume of the catalyst. Herein, GHSV values are provided as total volumes for both Catalyst and Catalyst Materials. This is a different reporting approach than what is being applied for WHSV, which is for the catalyst weight only, without support weight. Therefore, supported catalyst and unsupported catalysts will have similar WHSV but different GHSV. For supported catalysts, the GHSV reported is for the catalyst only, where the total volume of the catalyst material measured when multiplied by the wt. % of catalyst was calculated to be the volume of the catalyst.

For the MRU experiments, the mol. % ethane conversion temperature is determined at the WHSV of 5.46 h⁻¹, and a gas hourly space velocity (GHSV) in the range of 3000 to 7000 h⁻¹. The gaseous product exiting the catalyst bed is directed to vent during runs. When the gaseous product is to be analyzed, it is momentarily redirected to a gas chromatography unit to determine the percent of ethane, ethylene, O₂, CO₂, CO, and acetic acid. The gas exiting the reactor was analyzed by hot gas, gas chromatography (HGGC). Conversion (C) of the ethane feed gas was calculated as a volume flow rate change of ethane in the product compared to feed ethane mass flow rate using the following formula:

$$C = ( \frac{2 * {X}_{Ethylene} + {X}_{CO 2} + {X}_{C O} + 2 * {X}_{Acetic Acid}}{2 * {X}_{Ethylene} + 2 * {X}_{Ethane} + {X}_{CO 2} + {X}_{C O} + 2 * {X}_{Acetic Acid}} ) * 100 \% \qquad Eq . 3$$

In Eq. 3, X is the molar concentration of the corresponding compound in the gaseous effluent exiting the reactor at corresponding temperature.

Furthermore, the gas exiting the reactor was analyzed by GC to determine catalyst or catalyst material selectivity to ethylene (i.e., the percentage on a molar basis of ethane that forms ethylene). Selectivity to ethylene (S_{Ethylene}) was determined using the following equation:

$${S}_{Ethylene} = ( \frac{2 * {X}_{Ethylene}}{2 * {X}_{Ethylene} + {X}_{CO 2} + {X}_{C O} + 2 * {X}_{Acetic Acid}} ) * 100 \% \qquad Eq . 4$$

In the above equation, S_{Ethylene} is the selectivity to ethylene and X is the molar concentration of the corresponding compound in the gaseous effluent exiting the reactor at corresponding temperature.

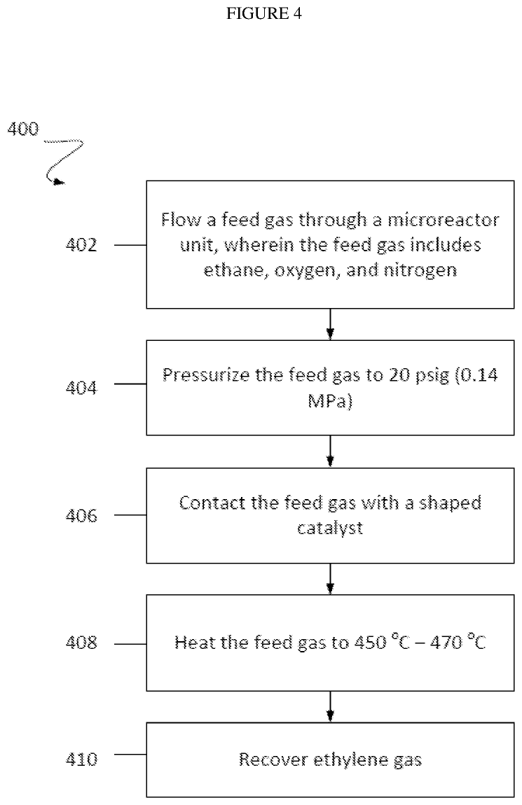

FIG. 4 is a flowchart of an example method 400 of the oxidative dehydrogenation of ethane. At 402, a feed gas that includes ethane, oxygen, and nitrogen is flowed through a microreactor unit. At 404, the feed gas is pressurized to 20 psig (0.14 MPa). In some implementations, the feed gas can be pressurized from 2-80 psig. At 406, the feed gas is contacted with a shaped catalyst as described herein. At 408, the feed gas is heated to 450° C.-470° C. At 410, ethylene gas is recovered.

The catalysts were analyzed with MRU short-term runs and MRU long-term runs. MRU short-term runs were conducted the day before a long-term run to determine ˜50 mol. % ethane conversion conditions. For the MRU short-term runs, the catalysts were tested on the MRU under several temperature conditions from 340-480° C., while the feed composition and weight hourly space velocity (WHSV) were held constant. At each temperature condition, gas samples were passed through a gas chromatogram (GC) to obtain the composition of the gas products. Conversion was calculated by percentage of atomic carbon in ethane product over atomic carbon in all carbon products including ethane, ethylene, carbon dioxide, and carbon monoxide, as shown in Eq. 3. Ethylene selectivity was calculated by percentage of ethane to ethylene at each temperature. The amount of acetic acid formed is small enough to exclude the amount of acetic acid from equations 3 and 4 when calculating conversion and selectivity.

Once the 50 mol. % ethane conversion temperature was determined in a short-term run, a long-term MRU experiment was be conducted at this temperature. The MRU would be elevated to the determined temperature and the process allowed to stabilize (usually about 30 minutes). After the process stabilizes, a GC injection was taken to confirm that the ethane conversion is close to 50 mol. % (±2%). If conditions were correct to achieve the 50 mol. % ethane conversion, then a MRU long-term run is initiated, otherwise the conditions (temperature) is adjusted to get the conversion closer to the target conversion. Once stable conversion and selectivity target conditions are met, the experimental timer starts for the long-term run.

Long-term MRU runs yield information on catalyst performance stability with increasing duration, or what is often referred to in heterogeneous catalysis as “time on stream” (TOS). Ideally, with extended duration of TOS, that catalyst conversion and selectivity percentages should stabilize and stay relatively unchanged throughout the duration of the experiment. It is not uncommon to have heterogeneous catalysts be more active (higher conversion percentages) within the first 24-72 hours, after which, the conversion stabilizes. However, the key trend to observe with a long-term run is that at some point in the experiment, the catalyst's conversion trend moves towards unchanging with increasing experimental time (flat or nearly flat line trend on x vs. y plot). Equally, if not more important, is that the ethylene selectivity also shows a stable trend of not decreasing with increasing TOS. MRU long term validation testing is done 42 days or 1008 hours of time on stream.

Catalysts can also exhibit deactivation over time in an MRU experiment or other catalytic processes. Advantageously, the shaped catalysts described herein have a very subtle deactivation trend, which only become obvious over long experiments (e.g. 300+ hours). The rate of deactivation can be expressed by mol. % ethane conversion absolute/10 hour. Accordingly, the catalysts described herein are stable over long experiments, and can be reused multiple times.

TABLE 1
Catalyst Formulations and Compositions for Examples 1-11

| | | | Wt. % | | Wt. % | | Wt. % |
|---|---|---|---|---|---|---|---|
| | | Support | Proportion | Binders | Proportion | Lubricant | Proportion |
| Shaped | Active | Manufacturer | of Support | Manufacturer | of Binders | Manufacturer | of Lubricant |
| Catalyst | Catalyst | and Product | to Total | and Product | to Total | and Product | to Total |
| No. | Phase | Identifier | Mixture | Identifier | Mixture | Identifier | Mixture |
| 1.2 | MoVTeTaOx | None | 0% | PVA (Mowiol 4-88) | 80/20 PVA/PEG | Natural | 2% |
| | | | | 1000 MW PEG | 2.35% PVA | graphite | |
| | | | | (Sigma PN 81189) | 0.59% PEG | (Sigma PN | |
| | | | | | | 1002969522) | |
| 1.3 | MoVTeTaOx | Fumed Silica | 58.2% | PVA (Mowiol 4-88) | 80/20 PVA/PEG | None | 0% |
| | | (Sigma PN | | 1000 MW PEG | 2.40% PVA | | |
| | | S5130) | | (Sigma PN 81189) | 0.60% PEG | | |
| 1.4 | MoVTeTaOx | Fumed Silica | 57.0% | PVA (Mowiol 4-88) | 80/20 PVA/PEG | Natural | 2% |
| | | (Sigma PN | | 1000 MW PEG | 2.35% PVA | graphite | |
| | | S5130) | | (Sigma PN 81189) | 0.59% PEG | (Sigma PN | |
| | | | | | | 1002969522) | |
| 1.5 | MoVTeTaOx | Fumed Silica | 57.0% | PVA (Mowiol 4-88) | 80/20 PVA/PEG | None | 0% |
| | | (Sigma PN | | 1000 MW PEG | 4.00% PVA | | |
| | | S5130) | | (Sigma PN 81189) | 1.00% PEG | | |
| 1.6 | MoVTeTaOx | Fumed Silica | 58.2% | PVA (Mowiol 4-88) | 60/40 PVA/PEG | None | 0% |
| | | (Sigma PN | | 1000 MW PEG | 1.80% PVA | | |
| | | S5130) | | (Sigma PN 81189) | 1.20% PEG | | |
| 1.7 | MoVTeTaOx | Fumed Silica | 57.0% | PVA (Mowiol 4-88) | 60/40 PVA/PEG | None | 0% |
| | | (Sigma PN | | 1000 MW PEG | 3.00% PVA | | |
| | | S5130) | | (Sigma PN 81189) | 2.00% PEG | | |
| 1.8 | MoVTeNbOx | Fumed Silica | 57.0% | PVA (Mowiol 4-88) | 60/40 PVA/PEG | None | 0% |
| | | (Sigma PN | | 1000 MW PEG | 3.00% PVA | | |
| | | S5130) | | (Sigma PN 81189) | 2.00% PEG | | |
| 2.1 | None | Fumed Silica | 95.0% | PVA (Mowiol 4-88) | 60/40 PVA/PEG | None | 0% |
| | | (Sigma PN | | 1000 MW PEG | 3.00% PVA | | |
| | | S5130) | | (Sigma PN 81189) | 2.00% PEG | | |
| 2.2 | None | Dry ball | 95.0% | PVA (Mowiol 4-88) | 60/40 PVA/PEG | None | 0% |
| | | milled Saint- | | 1000 MW PEG | 3.00% PVA | | |
| | | Gobain | | (Sigma PN 81189) | 2.00% PEG | | |
| | | Denstone-99 | | | | | |
| | | alpha- | | | | | |
| | | alumina | | | | | |

The reagents for the experiments described herein were purchased from manufacturers and used as received, without further purification. The following chemicals were purchased from Sigma Aldrich: fumed synthetic amorphous silica (Product No. S5130), natural graphite (Product No. 1002969522), MOWIOLR 4-88 31,000 MW Polyvinyl alcohol (Product No. 81381), 1000 MW Polyethylene glycol (Product No. 81189), and magnesium stearate (Product No. 415057). The following chemicals were acquired from Saint-Gobain: α-alumina (DENSTONE® 99), Silica XS 69025 catalyst support tablets (Sample No 2001910060), aluminum silicate SA 3X32 catalyst support tablets (Sample No 2002920068), and gamma-alumina SA 6X73 catalyst support tablets (Sample No 2007910008). The α-alumina (DENSTONE 99) was prepared for use through dry ball mill grinding using a RETSCH® PM100. Dry ball milling was performed using 3 mm zirconia balls and 500 mL zirconia grinding jar. The precipitated synthetic amorphous silica was obtained from PQ Corporation, product identifier PQ BRITESORB® C935.

In some implementations, a MoVTeTaOx catalyst active phase was prepared by dissolving (NH₄)₆Mo₇O₂₄·4H₂O(s) salt (2372.0 g; 1.9 mol) in 27 L of distilled water in a 50 L heated glass vessel at a temperature of 60° C., additional 3 L od distilled water was used for rinsing (portions: 1 L to rise powder+2 L after transfer of solution to SS reactor). The (NH₄)₆Mo₇O₂₄(aq) solution was clear and colorless and measured a pH of 5.04 at 55.0° C. Te(OH)₆(s) salt (514 g; 2.25 mol) was dissolved in 2 L of distilled water in a 20 L heated PFA vessel at a temperature of 55° C., additional 3 L of distilled water was used for rinsing (1 L+2 L portions). The Te(OH)₆(aq) solution was clear and colorless. The Te(OH)₆(aq) solution was transferred to the (NH₄)₆Mo₇O₂₄(aq) solution at 60° C. all at once. There was no visible change in the solution upon addition, however, the pH dropped to 3.20 at 53.0° C. After the (NH₄)₆Mo₆TeO₂₄ solution reached 55° C., 104.7 g of 28 wt. % NH₄OH (aq) was used to adjust the pH of the solution to pH 5.2 via dropwise addition. The final pH of 5.23 was measured at 56.9° C. The solution temperature was raised to 78° C., allowing 1 hour and 25 minutes for the solution to reach 78° C. The solution was allowed to stir for 1 hour at 78° C. The pH was re-measured as 4.91 at 78° C. before the solution was cooled back down to 60° C. The 60° C. solution was pumped into a 100 L, glass lined SS reactor vessel, which was pre-heated to skin temperature of 60° C. The VOSO₄·3.36H₂O salt (1989 g; 8.902 mol) was dissolved in 8 L of distilled H₂O in a heated Perfluoroalkoxy (PFA) 20 L vessel at a temperature of 55° C., additional 4 L of water was used for rinsing (2 L+2 L). The VOSO₄(aq) solution was a clear and vibrant blue color. Once homogeneous, the 55° C. VOSO₄(aq) solution was transferred at the controlled pump rate of 367 mL/min into 100 L SS reactor vessel to be mixed with the (NH₄)₆Mo₆TeO₂₄(aq) solution, while stirring at 22 rpm.

1.3 L of the tantalum reagent, (192 g of Ta₂O₅ and & 122 g of oxalate per 1 L of aqueous solution) was diluted with 2 L of distilled water in 20 L PFA vessel at room temperature. Additional 3 L of distilled water was used for rinsing (1 L+2 L). The tantalum oxalate solution was pumped to the MoTeV solution in SS reactor at the rate of 183 mL/min, while the reactor contents were being mixed at 22 rpm. Once the addition was completed, the reaction mixture was allowed to stir in air at 60° C. for 1 hour. The total volume of the water used for the reaction, including rising was 53 L. The reactor was sealed, and the head space was purged 4× with 10 psig nitrogen gas and vacuum alternating. The reactor was left sealed with a 20 psig nitrogen headspace. The back-pressure regulator was set to a setpoint of about 140 psig and the heat controller set to 165° C. The limit setting of the reactor's skin temperature was 195° C. After approximately 20 hours the slurry in the reactor reached 160° C. The time of the reaction was started from that point. The reaction time was 48 hours. The pressure registered inside the reactor reached maximum 103 psig. After 48 hours of reaction the heater for the reactor was turned off and the slurry was allowed to cool down to 68° C. while stirring was continued. The dark purple slurry was transferred to a filter unit, where the solids were filtered. The formed catalyst cake on the filter was rinsed with total 200 L of distilled water in portions of 40 L and 20 L.

This apparatus was attached to the reactor head but is a tube-in-tube exchanger (condenser) that had cooling water circulating on the outside tubing at ˜25° C. (controlled via closed system, cooling bath) and the inside tubing was connected to the reactor (process) to allow venting of excess gaseous pressure via a backpressure regulator.

The resulting solid cake was removed from the filter unit and transferred to an oven where it was dried in air at 90° C. for 4 days. The weight of the dried cake was 1937 g, which was 57% yield. The cake was ground into powder using RETSCH jaw crusher BB50 grinder.

The uncalcined cake was loaded in two quartz boats and the catalyst was air treated in an oven at 250° C. for 6 hours. After the air treatment the boats were transferred into a quartz tube of the Catalyst Calcining Furnace and purged with bulk nitrogen at 1000 sccm flow overnight. Two hours prior to starting the furnace, the bulk inlet nitrogen purge (<10 ppm oxygen) was redirected through an oxygen trap (LabClear Oxiclear™ gas purifier) to purge the tube with purified nitrogen (<1 ppm oxygen). The catalyst was calcined for 4 hours at 600° C. under purified nitrogen. The calcination was brought up to temperature with a 1.6° C./min ramp rate. After cooling to room temperature, the calcination yield was measured to be 93.5 wt. % recovery.

The final, calcined MoVTeTaOx is termed catalyst 1, herein. It may be understood that other procedures may be used to form catalyst 1. Further, any number of ratios of the metals may be used.

In some implementations, a MoVTeNbOx catalyst active phase was prepared by weighing 11.37 g of Nb₂O₅·H₂O and approximately 127 mL of de-ionized water into a 250 mL RBF. Added 19.09 g of oxalic acid (H₂C₂O₄) and stirred at 65° C. for 24 hours resulting in a turbid white solution of niobium oxalate.

Weighed 84.28 g of (NH₄)₆Mo₇O₂₄·4H₂O and approximately 200 mL of de-ionized water into a 500 mL round bottom flask (RBF), stirred to dissolve the powder. Weighed 18.27 g of telluric acid into a 100 ml beaker and approximately 50 mL of de-ionized water, the mixture was stirred at approx. 50° C. to dissolve the powder. The telluric acid solution was added to the (NH₄)₆Mo₇O₂₄·4H₂O solution dropwise at room temperature. Heated the Mo/Te solution to approximately 80° C. using an oil bath. 28.0 mL of NH₄OH solution was added dropwise to the Mo/Te solution to adjust the pH to 7.5 at 80° C.; the contents of the flask was left to stir at 80° C. for 2 hours. 28.0 mL of 5 M H₂SO₄ solution was added dropwise to the flask to adjust the pH to 4.99. The contents of the flask was stirred at 80° C. for approximately 2 hours

Weighed 70.04 g of VOSO₄·3.41H₂O and approximately 150 mL of de-ionized water into a 250 ml beaker; the mixture was stirred to dissolve the powder. In a 2 L RBF, the VOSO₄ solution was added dropwise using a dropping funnel to the Mo/Te solution at 70° C. and stirred for approximately 30 minutes, forming an opaque purple solution.

The previously prepared niobium oxalate solution was added slowly into the purple solution using a dropping funnel at 70° C. and stirred for approximately 30 minutes, forming a greenish slurry with a purple film on a walls of the RBF. The green slurry was transferred to a 2 L glass liner with a large magnetic stir bar.

The glass liner was placed inside a steel body of an autoclave and a cover was sealed to the steel body. The autoclave was slowly filled with nitrogen and then evacuated 10 times to remove air in the headspace. The autoclave was left overnight (about 10 to about 16 hours) at about 15 psig nitrogen and stirring at about 200 rpm. The nitrogen headspace pressure was released through a bubbler to purge the air out of the lines.

After stirring overnight, a backpressure regulator on the autoclave was set to 160 psig, the temperature controller was raised to 170° C. and a condenser water bath on an outline line was set to 25° C. The temperature profile was monitored. The reaction was left to stir at 200 rpm for an additional 17 hours at 165° C., for a total of about 24 hours.

The heat was turned off, top valve closed, insulation removed and the reactor was allowed to cool overnight while stirring at 200 rpm. The pressure in the reactor was released by opening the top valve.

The reactor was disassembled and the glass liner was removed. The contents were filtered using an aspirator through a Buchner funnel with four WHATMANR® #4 filter papers (available from Sigma Aldrich). The resulting filter cake was washed with de-ionized water until the filtrate was clear. The filter cake was placed in an oven at 90° C. to dry over a weekend (about 48 to about 60 hours) and allowed to cool to room temperature.

A portion of the dried filter cake was manually ground using a mortar and pestle. The ground catalyst was loaded the catalyst into boats and calcined at 600° C. under a nitrogen flow.

The final, calcined MoVTeNbOx is termed catalyst 2, herein. It may be understood that other procedures may be used to form catalyst 2. Further, any number of ratios of the metals may be used.

Distilled water was prepared in-house using a Corning Mega Pure™ 12A System ACS as distillation apparatus.

### Example 1: Granule Particle Size Distribution

The effect of granule particle size distribution on green body tablet strength was investigated. As described herein, the shaped catalyst formulations including an active catalyst phase, support, binder and optionally lubricant were prepared in a tablet press. Table 2 shows the granule size distribution used to study the impact of particle size on autopressed tablet's crush strength for Shaped Catalyst 1.4 (green bodies). The tablets in Table 2 were produced using a CPR-6 Automated press. First, 0.3616 g of polyethylene glycol (PEG) and 1.4437 g of polyvinyl chloride (PVA) were combined in a 1000 mL beaker. Next, 80 mL of distilled water was added to the beaker. The beaker was placed in a 65° C. water bath on a stirring hot plate to dissolve the chemicals while stirring. After the chemicals dissolved, 24.0622 g of catalyst 1 and 36.16 g of fumed silica were combined in a separate container, and then added to the 1000 ml beaker with dissolved PVA/PEG. An additional 160 mL of distilled water was added to form a runny slurry. The water bath setting was increased to 95° C. An overhead stirrer stirred the slurry until most of the water evaporated and the mixture formed a thick paste. The beaker including the paste was placed into an oven at 105° C. overnight to dry completely.

The formulated catalyst was then granulated in by creating a briquette, crushing the briquette, sieving the formed granules, and collecting the granules with dimensions within the size range of (>105 μm and <710 μm). Before briquetting, the formulated solid catalyst was first powdered in a mortar and pestle. About 7 g of the powder was loaded into 4 cm diameter die set and pressed on a CARVER manual press to 12 metric tons for 60 seconds to create a briquette. The briquette was then crushed using mortar and pestle to yield granules. The granules were sieved using sieves sized 105 μm, 250 μm, 500 μm, and 710 μm. The granules collected on sieves 105 μm to 500 μm were collected for further experiments (size range >105 μm and <710 μm). The process of granulating was repeated several times until satisfactory amount of granules was obtained. Table 2 shows the granule size distributions used in this experiment. The granules were then tableted into 3 mm tablets. The granulated catalyst sample was mixed with 2 wt. % graphite as a lubricant, and was then tableted using the CPR-6 automatic single punch tablet press

The tablet press settings for all above samples were: top die pressure at 8, bottom die setting at 2, speed of tableting at 4. Axial crush strength was determined by applying a load slowly to the axial, i.e., flat sides, of the cylindrical tablet, to a tablet sandwiched between two flat-faced plates. The highest force recorded when the tablet breaks is recorded as the crush strength. The apparatus used to determined crush strengths included a CHATILLON® TCM 201 drive and a MECMESIN® AFG 2500N force gauge, which is mounted on the drive.

Comparing the crush strength values of the Shaped Catalysts 1.4, 1.4-E1, 1.4-E2, and 1.4-E3, it was found that E2 with a particle size distribution (x) of 250 μm<x<500 μm achieved the highest crush strengths.

TABLE 2
Granule Particle Size Distribution for Crush Strength Experiments

| Shaped | Granule Particle | | Mean Crush | Standard |
|---|---|---|---|---|
| Catalyst | Size Distribution | Axial Crush | Strength | Deviation |
| No. | (μm) | Strengths (N) | (N) | (N) |
| 1.4 | 105-710 | 89, 9, 126, 42, | 55 | 40 |
| | | 75, 8, 36, 55 | | |
| 1.4-E1 | 150-250 | 92, 136, 77, 75, | 62 | 40 |
| | | 79, 53, 2, 48, | | |
| | | 57, 2 | | |
| 1.4-E2 | 250-500 | 163, 158, 105, | 102 | 54 |
| | | 142, 2, 94, 36, | | |
| | | 71, 100, 147 | | |
| 1.4-E3 | 500-710 | 20, 60, 53, 76, | 43 | 33 |
| | | 39, 2, 2, 7, | | |
| | | 25, 86, 2 | | |

### Example 2: Tablet Processability Data of Shaped Catalyst 1.5 Tablets

In addition to the axial strength measurements on 3 mm Shaped Catalyst 1.4 tablets, the radial crush strength of a 6 mm Shaped Catalyst 1.5 (green body, GB) tablet was investigated. The tablets were prepared on an autopress. The Shaped Catalyst 1.5 (GB) tablets were prepared by combining 0.4009 g of polyethylene glycol (PEG) and 1.6159 g of polyvinyl chloride (PVA) in a 500 ml beaker. Next, 60 mL of distilled water was added to the beaker. The beaker was placed in a 65° C. water bath on a stirring hot plate to dissolve the chemicals while stirring. After the chemicals dissolved, 16.0457 g of catalyst active phase catalyst 1 and 24.0425 g of fumed silica were weighed and combined in a separate container. Next, the catalyst/silica mixture was added to the 500 ml beaker with dissolved PVA/PEG. An additional 150 mL of distilled water was added to form a runny slurry. The water bath was increased to 95° C. The slurry was stirred using an overhead stirrer until most of the water evaporated and the mixture formed a thick paste. The beaker with the paste was placed into an oven at 105° C. overnight to dry completely.

The formulated solid catalyst was next powdered in a mortar and pestle. About 7 g of the powder was loaded into 4 cm diameter die set and pressed on the CARVER manual press to 12 metric tons for 60 seconds to create a briquette. The briquette was then crushed using mortar and pestle to granules. The granules were sieved using sieves sized 250 μm and 500 μm. The granules collected on sieve 250 μm were collected for further experiments (size range >250 μm and <500 μm). The process of granulating was repeated several times until satisfactory weight of granules was obtained. The granules were used for tableting.

The granulated catalyst was tableted using the CPR-6 automatic single punch tablet press. The settings of the instrument were: top die setting at 8, bottom die setting at 2, speed of tableting varied at between 1 and 3. Tableting produced the Shaped Catalyst 1.5 (green body, GB).

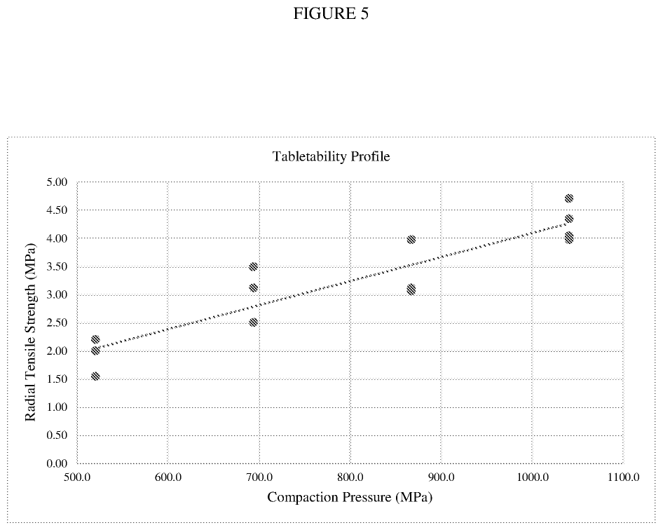

FIG. 5 shows the tabletability profile of the Shaped Catalyst 1.5 green body tablets (Shaped Catalyst 1.5 GB). The tested tablets were 6 mm in diameter. The radial crush strength (in MPa) was tested at various compaction pressures along the diameter of the cylindrical tablet by applying a load slowly to a tablet sandwiched between two flat-faced plates. The highest force recorded when the tablet breaks is recorded as the radial crush strength. The apparatus used to determined crush strengths included a CHATILLON TCM 201 drive and a MECMESIN AFG 2500N force gauge, which is mounted on the drive. The radial crush strength was converted into tensile strength values as described herein.

As shown in FIG. 5, the Shaped Catalyst 1.5 GB tablets displayed increasing radial tensile strength at increasing compaction pressures. The increasing radial tensile strength with increasing compaction pressure of Catalyst tablets 1.5 GB shows that these tablets are well suited for tablet pressing. Additionally, these tablets have a radial tensile strength above 2 MPa, further indicating their suitability as catalysts.

### Example 3: Tablet Processability Data of Shaped Catalyst 1.7 Tablets

Shaped Catalyst 1.7 tablets were prepared by combining 0.8079 g of polyethylene glycol (PEG) and 1.1960 g of polyvinyl chloride (PVA) in a 1000 ml beaker. Next, 100 mL of distilled water was added to the beaker. The beaker was placed in a 65° C. water bath on a stirring hot plate to dissolve the chemicals while stirring. After the chemicals dissolved, 16.0320 g of catalyst 1 and 24.05 g of fumed silica were combined in a separate container, and then added to the 1000 ml beaker with dissolved PVA/PEG. An additional 150 mL of DI water was added to form a runny slurry. The water bath was increased to 95° C. An overhead stirrer stirred the slurry until most of the water evaporated and the mixture formed a thick paste. The beaker with the paste was placed into an oven at 105° C. overnight to dry completely.

The formulated solid catalyst was then powdered in a mortar and pestle. About 7 g of the powder was loaded into 4 cm diameter die set and pressed on the CARVER manual press to 12 metric tons for 60 seconds to create a briquette. The briquette was then crushed using mortar and pestle to create granules. The granules were sieved using sieves sized 250 μm and 500 μm. The granules collected on the 250 μm sieve were collected for further experiments (size range >250 μm and <500 μm). The process of granulating was repeated several times until a sufficient amount of granules was obtained for tableting.

The granulated catalyst was tableted using the CPR-6 automatic single punch tablet press. The settings of the instrument were: top die setting at 8, bottom die setting at 2, speed of tableting varied at between 1 and 3. Tableting produced the Shaped Catalyst 1.7 (green body, GB).

The radial crush strengths of Shaped Catalyst 1.7 GB tablets were tested at various compaction pressures along the diameter of the cylindrical tablet by applying a load slowly to a tablet sandwiched between two flat-faced plates. The highest force recorded when the tablet breaks was recorded as the radial crush strength. The apparatus used to determine crush strengths included a CHATILLON TCM 201 drive and a MECMESIN AFG 2500N force gauge, which is mounted on the drive. The radial crush strength is converted into tensile strength values as described herein.

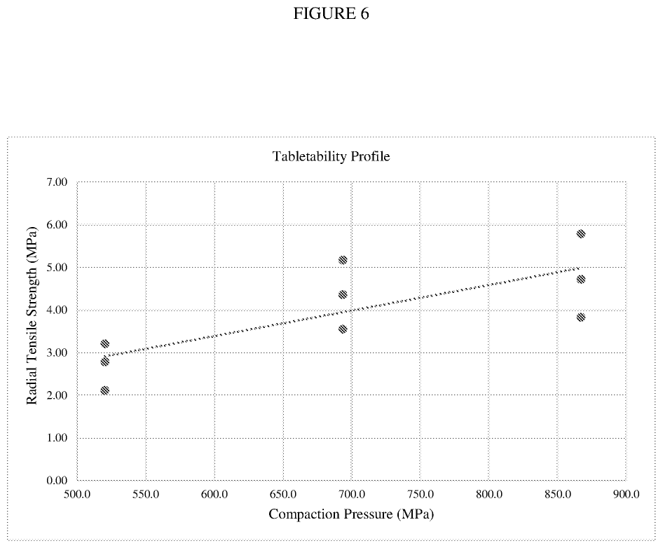

FIG. 6 shows the tabletability profile, i.e., the radial tensile strength in MPa as a function of compaction pressure in MPa, of the Shaped Catalyst 1.7 GB tablets. As shown in FIG. 6, the Shaped Catalyst 1.7 GB tablets displayed increasing radial tensile strength at increasing compaction pressures. The increasing radial tensile strength with increasing compaction pressure of Catalyst tablets 1.7 GB shows that these tablets are well suited for tablet pressing. Additionally, these tablets have a radial tensile strength above 2 MPa, further indicating their suitability as catalysts.

As shown in Experiments 2 and 3, the crush strengths of shaped catalyst tablets with 5 wt. % of binder (1.5 GB) were approximately 63% higher than those prepared with ˜3 wt. % of binder (1.4-E2-GB). Accordingly, the shaped catalyst green body tablets prepared with higher binder content had more than twice the crush strength of those prepared with lower binder content. This was a surprising result.

### Example 4: Tabletability Profile of Comparative Examples 2.1 and 2.2

In order to evaluate catalyst formation tabletability profiles, comparative examples were analyzed. These examples contained support and binder ingredients but no catalyst. The two comparative formulations including alpha-alumina (Shaped Catalyst 2.1) and fumed silica (Shaped Catalyst 2.2) support materials. The comparative examples Shaped Catalyst 2.1 and 2.2 were formulated as shown in Table 1.

For Shaped Catalyst 2.1 comparative example tablets, 0.8143 g of polyethylene glycol (PEG) and 1.2111 g of polyvinyl chloride (PVA) were combined in a 1000 mL beaker. Next, 50 mL of distilled water was added to the beaker. The beaker was placed in a 65° C. water bath on a stirring hot plate to dissolve the chemicals while stirring. After the chemicals dissolved, 39.9 g of fumed silica was added to the 1000 ml beaker with dissolved PVA/PEG. An additional 120 mL of distilled water was added to form a runny slurry. The water bath was increased to 95° C. An overhead stirrer stirred the slurry until most of the water evaporated and the mixture formed a thick paste. The beaker with the paste was placed into an oven at 105° C. overnight to dry completely.

The resulting dry “cake” was crushed in mortar and pestle into granules, but it was not pressed into a briquette as the other samples. The granules were sieved using sieves sized 250 μm and 500 μm. The granules collected on the sieve size 250 μm were collected for further experiments (size range >250 μm and <500 μm). The process of granulating was repeated several times until satisfactory weight of granules was obtained. The granules were used for tableting. The granulated catalyst was tableted using the CPR-6 automatic single punch tablet press. The sample was manually loaded into the press die.

For Shaped Catalyst 2.2 comparative example tablets, 0.7976 g of polyethylene glycol (PEG) and 1.2018 g of polyvinyl chloride (PVA) were combined in a 350 ml beaker. Next, 50 mL of distilled water was added to the beaker. The beaker was placed in a 65° C. water bath on a stirring hot plate to dissolve the chemicals while stirring. After the chemicals dissolved, 40.1682 g of dry ball milled DENSTONE 99 alpha-alumina was added to the 350 ml beaker with dissolved PVA/PEG. A runny slurry formed. The temperature of the water bath was increased to 95° C. An overhead stirrer stirred the slurry until most of the water evaporated and the mixture formed a thick paste. The beaker with the paste was placed into an oven at 105° C. overnight to dry completely.

After drying overnight, the paste becomes a dry “cake”, i.e., an agglomerated solid powder that can be ground or shaped. The resulting solid powder was crushed in mortar and pestle into granules, but it was not pressed into a briquette as the other samples. The granules were sieved using sieves sized 250 μm and 500 μm. The granules collected on the sieve size 250 μm were collected for further experiments (size range >250 μm and <500 μm). The process of granulating was repeated several times until a sufficient amount was obtained for tableting.

The granulated catalyst was tableted using the CPR-6 automatic press. The sample was manually loaded into the press die.

The radial crush strengths of the comparative example Shaped Catalysts 2.1 GB and 2.2 GB tablets were tested at various compaction pressures along the diameter of the cylindrical tablet by applying a load slowly to a tablet sandwiched between two flat-faced plates. The highest force recorded when the tablet breaks was recorded as the radial crush strength. The apparatus used to determined crush strengths included a CHATILLON TCM 201 drive and a MECMESIN AFG 2500N force gauge, which is mounted on the drive. The radial crush strength values were converted into tensile strength values as described herein.

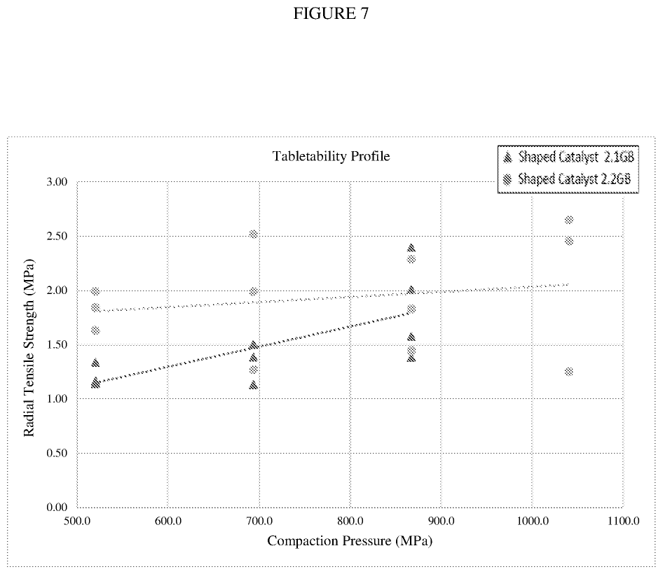

FIG. 7 shows the tabletability profile of the Shaped Catalyst 2.1 GB comparative example tablets and Shaped Catalyst 2.2 GB comparative example tablets (circles). The tested tablets were 6 mm in diameter. As shown in FIG. 7, Shaped Catalyst 2.2 GB comparative example tablets did not exhibit increasing radial tensile strength with increasing compaction pressure, suggesting that fumed silica on its own is not an ideal tablet pressing material. Rather, the fumed silica support and catalyst active phase work synergistically to improve the strength and tabletability of the shaped catalysts tablets. This was another surprising result.

Shaped Catalyst 2.1 GB comparative example tablets did exhibit increased radial tensile strength at increased compaction pressures, however, Shaped Catalyst 2.1 GB comparative example tablets include an alpha-alumina support phase.

Without wishing to be bound by theory, it appears that when a MoVTeTaOx catalyst active phase is combined with fumed silica, the new material mixture behaves like an ideal pressable material, whereas fumed silica formulations without the catalyst do not show these characteristics. In addition, the combination of fumed silica and MoVTeTaOx catalyst active phase produces a material that presses better than established support such as alpha-alumina.

### Example 5: Mechanical Strength Data

The mechanical strength of the Shaped Catalyst 1.2-1.8 tablets as green bodies and as sintered bodies was analyzed. Table 3 lists the granule size of the formulations used to create the Shaped Catalyst 1.2-1.8 for this experiment.

TABLE 3
Granule Particle Size Distribution for Shaped Catalysts 1.1-2.2

| Shaped Catalyst No. | Granule Particle Size Distribution (μm) |
|---|---|
| 1.2 | 105-710 |
| 1.3 | 105-710 |
| 1.4 | 105-710 |
| 1.5 | 250-500 |
| 1.6 | 250-500 |
| 1.7 | 250-500 |
| 1.8 | 250-500 |

### Synthesis of Shaped Catalyst 1.2

Shaped Catalyst 1.2 tablets were synthesized by combining 0.0824 g of polyethylene glycol (PEG) and 0.3473 g of polyvinyl chloride (PVA) in a 100 ml beaker. Next, 10 mL of distilled water was added to the beaker. The beaker was placed in a 65° C. water bath on a stirring hot plate to dissolve the chemicals while stirring. In a separate 500 ml beaker, 5.7040 g of catalyst active phase catalyst 1 and 8.3985 g of fumed silica were combined. After the PGA/PVA mixture dissolved, it was combined with the catalyst/silica mixture. The 500 ml beaker with all four chemicals mixed together was placed in a water bath set to 95° C. An overhead stirrer stirred the slurry until most of the water evaporated and the mixture formed a thick paste. The beaker containing the paste was placed in an oven at 105° C. overnight to dry completely.

The formulated solid catalyst was then powdered in a mortar and pestle. About 7 g of the powder was loaded into 4 cm diameter die set and pressed on the CARVER manual press to 12 metric tons for 60 seconds to create a briquette. The briquette was then crushed using a mortar and pestle. The resulting granules were sieved using sieves sized 105 μm, 250 μm, 500 μm and 710 μm. The granules collected on sieves 105 μm to 500 μm were collected for further experiments (size range >105 μm and <710 μm). The process of granulating was repeated several times until a sufficient amount was obtained for tableting.

For tableting, 2 wt. % of natural graphite was added to the granules as a lubricant and mixed very well together. The granulated catalyst was tableted using the CPR-6 automatic single punch tablet press. The settings of the instrument were: top die pressure at 8, bottom die setting at 1, speed of tableting at 4. Tableting produced a green body tablet.

For the sintered body experiments, the green body tablets were further debinded and sintered. Debinding occurred on a Catalyst Calcination Furnace (CCF). The tablets were loaded into a quartz boat and the boat was inserted into a quartz tube of the CCF. The tube was sealed except for inlet and outlet lines, which were used to flow purified air through the quartz tube during the process at 300 sccm. Purified air is devoid of impurities, for example bottled air or Zero Air. Zero Air is air that has had hydrocarbons removed via a process of oxidative catalysis such that it only contains less than 0.1 parts per million of total hydrocarbons. The furnace was heated to 400° C. at the rate of 1° C./min, held at 400° C. for 1 hour, and then the heat was turned off and the furnace allowed to cool.

After the furnace cooled to room temperature, the process of sintering was started in the same furnace. The flow of air was switched to an N₂ flow at 300 sccm. The shaped catalysts were purged with N₂ at room temperature for at least 6 hours. After purging, the furnace was heated to 600° C. at a rate of 1.6° C./min in continuous N₂ flow, held at 600° C. for 2 hours, and then allowed to cool naturally to room temperature. The resulting shaped catalyst tablet was then removed for analysis.

### Synthesis of Shaped Catalyst 1.3

Shaped Catalyst 1.3 tablets were synthesized by combining 0.1236 g of polyethylene glycol and 0.4870 g of polyvinyl chloride in a 400 mL beaker. Next, 50 mL of distilled water was added to the beaker. The beaker was placed in a 65° C. water bath on a stirring hot plate to dissolve the chemicals while stirring. After the chemicals dissolved, 8.0356 g of catalyst active phase catalyst 1 and 12.0590 g of fumed silica were mixed together and then added to the same beaker to combine with the other two chemicals. An additional 40 mL of distilled water was added to form a runny slurry. The water bath setting was increased to 95° C. An overhead stirrer stirred the slurry until most of the water evaporated and the mixture became a thick paste. The beaker with the paste was placed into an oven at 105° C. overnight to dry completely.

The formulated solid catalyst was then granulated using the same procedure as used for Shaped Catalyst 1.2. The resulting granules were tableted. The granulated catalyst was tableted using the CPR-6 automatic press. The settings of the instrument were top die pressure at 8, bottom die setting at 1. Speed of tableting at 5 for both. The target was to get two types of tablets: with lower and higher crush strength, for future experiments to see if those catalyst tablets would have different activity. Tableting resulting in a green body tablet.

For the sintered body experiments, the green body tablets were further debinded and sintered. Debinding and sintering of Shaped Catalysts 1.3 was done using the same procedure as Shaped Catalysts 1.2.

### Synthesis of Shaped Catalyst 1.4-E2

The synthesis of Shaped Catalysts 1.4-E2 Tablets was performed using the same procedure as described in Example 1. For the sintered body experiments, the green body tablets were further debinded and sintered using the same procedure as Shaped Catalysts 1.2.

### Synthesis of Shaped Catalyst 1.5

The synthesis of Shaped Catalysts 1.5 was performed using the same procedure as described in Example 2. For sintered body experiments, the green body tablets were further debinded and sintered. Debinding and sintering of Shaped Catalysts 1.5 was done using the same procedure as Shaped Catalysts 1.2.

### Synthesis of Shaped Catalyst 1.6

Shaped Catalysts 1.6 were synthesized by combining 0.8061 g of polyethylene glycol (PEG) and 1.2062 g of polyvinyl chloride (PVA) in a 1000 ml beaker. Next, 100 mL of distilled water was added to the beaker. The beaker was placed in a 65° C. water bath on a stirring hot plate to dissolve the chemicals while stirring. After the chemicals dissolved, 16.0443 g of catalyst active phase catalyst 1 and 24.0130 g of fumed silica were combined in a separate container, and then the catalyst/silica mixture was added to the 1000 mL beaker with dissolved PVA/PEG. An additional 120 mL of distilled water was added to form a runny slurry. The water bath setting was increased to 95° C. An overhead stirrer stirred the slurry until most of the water evaporated and the mixture formed a thick paste. The beaker with the paste was placed into an oven at 105° C. overnight to dry completely.

The resulting dry “cake” was then powdered in a mortar and pestle. About 7 g of the powder was loaded into a 4 cm diameter die set and pressed on the CARVER manual press to 12 metric tons for 60 seconds to create a briquette. The briquette was then crushed using mortar and pestle to form granules. The granules were sieved using sieves sized 250 μm and 500 μm. The granules collected on sieve 250 μm were collected for further experiments (size range >250 μm and <500 μm). The process of granulating was repeated several times until a sufficient amount was obtained for tableting.

The granulated catalyst was tableted using the CPR-6 automatic single punch tablet press. The settings of the instrument were: top die setting at 8, bottom die setting at 2, speed of tableting varied at between 1 and 3.

For the sintered body experiments, the green body tablets were further debinded and sintered using the same procedure as Shaped Catalysts 1.2.

### Synthesis of Shaped Catalyst 1.7

The synthesis of Shaped Catalysts 1.7 was performed using the same procedure as described in Example 3. For sintered body experiments, the green body tablets were further debinded and sintered using the same procedure as Shaped Catalysts 1.2.

### Synthesis of Shaped Catalyst 1.8

Shaped Catalysts 1.8 tablets were synthesized by combining 0.8099 g of polyethylene glycol (PEG) and 1.1990 g of polyvinyl chloride (PVA) in a 1000 ml beaker. Next, 50 mL of distilled water was added to the beaker. The beaker was placed in a 65° C. water bath on a stirring hot plate to dissolve the chemicals while stirring. After the chemicals dissolved, 16.0320 g of catalyst active phase catalyst 2 and 24.00 g of fumed silica were combined in a separate container, and then added to the 1000 ml beaker with dissolved PVA/PEG. An additional 200 mL of distilled water was added to form a runny slurry. The water bath setting was increased to 95° C. An overhead stirrer stirred the slurry until most of the water evaporated and the mixture formed a thick paste. The beaker with the paste was placed into an oven at 105° C. overnight to dry completely.

The resulting solid powder was then powdered in a mortar and pestle. About 7 g of the powder was loaded into 4 cm diameter die set and pressed on the CARVER manual press to 12 metric tons for 60 seconds to create a briquette. The briquette was then crushed using mortar and pestle to granules. The granules were sieved using sieves sized 250 μm and 500 μm. The granules collected on sieve 250 μm were collected for further experiments (size range >250 μm and <500 μm). The process of granulating was repeated several times until a sufficient amount was obtained for tableting.

The granulated catalyst was tableted using the CPR-6 automatic single punch tablet press. The settings of the instrument were: top die setting at 8, bottom die setting at 2, speed of tableting varied at between 1 and 3.

For sintered body experiments, the green body tablets were further debinded and sintered using the same procedure as described for Shaped Catalysts 1.2.

### Mechanical Strength Experiments

Two mechanical strength measurements were collected, axial crush strength and radial crush strength, and both are reported in Newtons (N). Axial crush strengths and radial crush strengths were obtained using the same procedure as described in Experiments 1-3. The axial crush strengths (in Newtons, N) of the green bodies and sintered bodies are shown in Table 4. The radial crush strengths of the sintered bodies are shown in Table 5.

As shown in Table 4, the shaped catalysts described herein have high mechanical strength as green bodies and as sintered bodies. The tablets containing approximately 3% binder (Shaped Catalysts 1.3, 1.4, and 1.6) showed increased mechanical strength after sintering, regardless of whether the binder included 80/20 PVA/PEG or 60/40 PVA/PEG. For the tablets containing approximately 5% binder (Shaped Catalysts 1.5, 1.7, and 1.8), the tablets containing a 60/40 ratio of PVA/PEG displayed increased mechanical strength after sintering.

Further, as shown in Tables 4 and 5, the shaped catalyst including the MoVTeNbOx active phase showed high mechanical strength as a green body and as a sintered body. This was a surprising result, and demonstrates that there is a synergistic effect between the MoVTeNbOx active phase and the fumed silica support.

The tabletability profiles show that the formulations are suitable for processing through tablet pressing equipment.

TABLE 4
Axial Crush Strength Data for 3 mm Green Body (GB) and Sintered
Body (SB) MoVTeTaOx Shaped Catalyst Tablets 1.2-1.8

| | Green Body | | Sintered Body | |
|---|---|---|---|---|
| | Mean | | Mean | |
| | Crush | Standard | Crush | Standard |
| | Strength | Deviation | Strength | Deviation |
| Catalyst Identifier | (N) | (N) | (N) | (N) |
| MoVTeTaOx Catalyst | 275 | 162 | 117 | 60 |
| Tablets 1.2 | | | | |
| MoVTeTaOx Shaped | 97 | 44 | 134 | 80 |
| Catalyst Tablets | | | | |
| 1.3SB | | | | |
| MoVTeTaOx Shaped | 102 | 54 | 131 | 59 |
| Catalyst Tablets | | | | |
| 1.4-E2-SB | | | | |
| MoVTeTaOx Shaped | 300 | 44 | 281 | 68 |
| Catalyst Tablets | | | | |
| 1.5SB | | | | |
| MoVTeTaOx Shaped | 128 | 9 | 135 | 52 |
| Catalyst Tablets | | | | |
| 1.6SB | | | | |
| MoVTeTaOx Shaped | 351 | 57 | 477 | 66 |
| Catalyst Tablets | | | | |
| 1.7SB | | | | |
| MoVTeNbOx Shaped | 105 | 17 | 117 | 60 |
| Catalyst Tablets | | | | |
| 1.8SB | | | | |

TABLE 5
Radial Crush Strength for 3 mm Sintered Body (SB) Support
Catalyst Tablet Samples 1.5SB, 1.7SB, and 1.8SB

| | | Mean Radial | Standard |
|---|---|---|---|
| | Radial Crush | Crush | Deviation |
| Catalyst Identifier | Strengths (N) | Strength (N) | (N) |
| MoVTeTaOx Shaped | 85, 49, 51, 64, 80, | 57 | 15 |
| Catalyst Tablets 1.5SB | 50, 64, 35, 40, 53 | | |
| MoVTeTaOx Shaped | 67, 64, 60, 37, 55, | 51 | 13 |
| Catalyst Tablets 1.7SB | 31, 70, 37, 46, 46 | | |
| MoVTeNbOx Shaped | 28, 41, 27, 28, 29, | 28 | 5 |
| Catalyst Tablets 1.8SB | 21, 22, 30, 23, 32 | | |

### Example 6: Apparent Density Data

The apparent density of the catalyst tablets described herein illustrates the impact of binder weight percentage on catalyst densification, as well as showcasing how increasing density is related to increasing crush strength. Table 6 shows the tablet dimensions, weight, and apparent density data for 3 mm sintered body shaped catalyst tablets 1.4-E1-SB, 1.4-E2-SB, 1.4-E3-SB, and 1.5SB-1.8SB. The sintered body tablets were prepared as described in Experiments 1-5. For each analysis, five tablets were analyzed.

As shown herein, shaped catalyst tablet 1.5 SB has higher density than Catalyst Tablets 1.6SB and shows that the 60/40 PVA/PEG binder combination was not more potent than an 80/20 PVA/PEG mixture when loaded in 3 wt. %. The more important factor is the increase in binder content from 3 wt. % to 5 wt. %. Shaped catalyst tablets 1.5 and shaped catalyst tablets 1.7 are both 5 wt. % binder, with different PVA/PEG, ratios but produce similar densities.

Shaped catalyst tablet 1.8 is a change in catalyst phase (MoVTeNbOx vs. MoVTeTaOx) but the same formulation of fumed silica/binder as shape catalyst tablet 1.7. This shows the catalyst active phase plays a part in the synergistic effect of the overall mixture pressability/mechanical strength/density. Further, density and crush strength (i.e., mechanical strength) are directly related from the data given. As shown herein, a higher density equates to a higher crush strength.

TABLE 6
Dimensions, Weight, and Density of 3 mm Sintered
Body Support Catalyst Tablet Samples

| | | | | Diam- | | Apparent |
|---|---|---|---|---|---|---|
| | | Weight | Height | eter | Volume | Density |
| Sample | | (g) | (mm) | (mm) | (mm³) | (g/cm³) |
| Supported | 1 | 0.03073 | 3.48 | 3.04 | 25.25 | 1.22 |
| Catalyst | 2 | 0.02785 | 3.56 | 2.99 | 24.98 | 1.11 |
| Tablets | 3 | 0.02952 | 3.53 | 3.00 | 24.94 | 1.18 |
| 1.4-E1-SB | 4 | 0.02300 | 3.46 | 3.01 | 24.61 | 0.93 |
| | 5 | 0.02316 | 3.46 | 2.99 | 24.28 | 0.95 |
| | AVG | 0.02685 | 3.50 | 3.01 | 24.81 | 1.08 |
| Supported | 1 | 0.02554 | 2.97 | 3.00 | 20.98 | 1.22 |
| Catalyst | 2 | 0.02495 | 2.98 | 2.98 | 20.77 | 1.20 |
| Tablets | 3 | 0.02824 | 3.21 | 2.99 | 22.53 | 1.25 |
| 1.4-E2-SB | 4 | 0.02810 | 3.20 | 3.02 | 22.93 | 1.23 |
| | 5 | 0.02495 | 3.01 | 3.00 | 21.27 | 1.17 |
| | AVG | 0.02636 | 3.07 | 3.00 | 21.70 | 1.21 |
| Supported | 1 | 0.02143 | 3.15 | 3.01 | 22.40 | 0.96 |
| Catalyst | 2 | 0.01919 | 3.15 | 3.03 | 22.70 | 0.85 |
| Tablets | 3 | 0.02042 | 3.20 | 3.00 | 22.61 | 0.90 |
| 1.4-E3-SB | 4 | 0.02171 | 3.18 | 3.01 | 22.62 | 0.96 |
| | 5 | 0.02151 | 3.21 | 3.02 | 22.98 | 0.94 |
| | AVG | 0.02085 | 3.18 | 3.01 | 22.66 | 0.92 |
| Supported | 1 | 0.02964 | 2.88 | 3.04 | 20.89 | 1.42 |
| Catalyst | 2 | 0.02955 | 2.98 | 3.00 | 21.05 | 1.40 |
| Tablets | 3 | 0.03202 | 3.02 | 3.06 | 22.20 | 1.44 |
| 1.5SB - 3 mm | 4 | 0.02988 | 3.00 | 3.03 | 21.62 | 1.38 |
| | 5 | 0.02989 | 2.94 | 3.02 | 21.05 | 1.42 |
| | AVG | 0.03020 | 2.96 | 3.03 | 21.36 | 1.41 |
| Supported | 1 | 0.02426 | 2.95 | 3.02 | 21.12 | 1.15 |
| Catalyst | 2 | 0.02913 | 3.22 | 3.03 | 23.21 | 1.26 |
| Tablets | 3 | 0.02790 | 3.23 | 3.02 | 23.13 | 1.21 |
| 1.6SB - 3 mm | 4 | 0.02655 | 3.25 | 2.98 | 22.66 | 1.17 |
| | 5 | 0.02802 | 3.18 | 3.05 | 23.22 | 1.21 |
| | AVG | 0.02717 | 3.17 | 3.02 | 22.67 | 1.20 |
| Supported | 1 | 0.03368 | 3.21 | 3.06 | 23.59 | 1.43 |
| Catalyst | 2 | 0.03291 | 3.15 | 3.05 | 23.00 | 1.43 |
| Tablets | 3 | 0.03316 | 3.18 | 3.07 | 23.53 | 1.41 |
| 1.7SB - 3 mm | 4 | 0.03285 | 3.18 | 3.07 | 23.53 | 1.40 |
| | 5 | 0.03423 | 3.22 | 3.06 | 23.67 | 1.45 |
| | AVG | 0.03337 | 3.19 | 3.06 | 23.46 | 1.42 |
| Supported | 1 | 0.02300 | 2.89 | 2.88 | 18.82 | 1.22 |
| Catalyst | 2 | 0.02550 | 2.85 | 2.96 | 19.60 | 1.30 |
| Tablets | 3 | 0.02290 | 2.63 | 2.97 | 18.21 | 1.26 |
| 1.8SB - 3 mm | 4 | 0.02560 | 2.97 | 2.96 | 20.43 | 1.25 |
| | 5 | 0.02630 | 2.95 | 3.07 | 21.83 | 1.21 |
| | AVG | 0.02466 | 2.85800 | 2.96800 | 19.78 | 1.25 |

### Example 7: MRU Data, Comparative Example MoVTeTaOx Catalyst-Only Shapes

MRU data provides the basis for interpreting if the shaped catalysts yield heterogeneous catalysis with robust, long-term performance with no or minimal losses in activity and/or selectivity. Losses can be regenerated by the appropriate means, but the time between regenerations should be as long as possible.

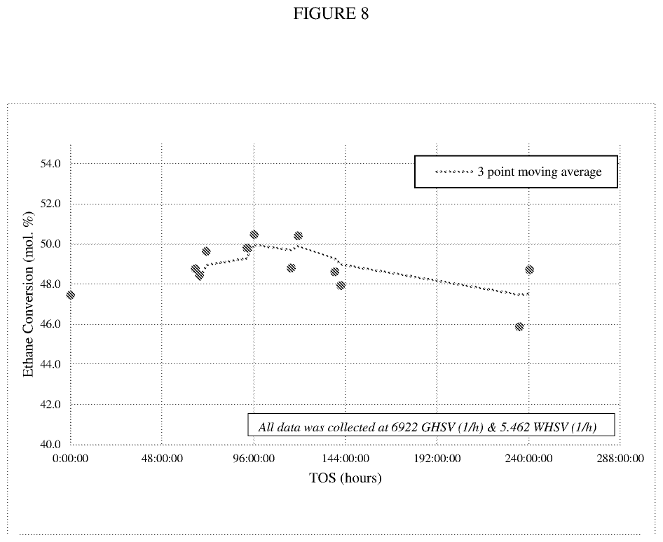

In order to evaluate the performance of the supported, shaped catalysts, a baseline was established with an unsupported, shaped catalyst. FIG. 8 shows MRU data of the long-term ethane to ethylene conversion for the MoVTeTaOx catalyst-only Shaped Catalyst 1.2SB tablets. The Shaped Catalyst 1.2SB tablets were prepared as described in Experiment 5 and the MRU experiment was conducted as described in the detailed description, with the following parameters.

The experimental results shown in FIG. 8 were collected at 470° C., 6922 GHSV hr⁻¹, and 5.462 WHSV hr⁻¹. The MRU procedure occurred as described herein. For the Shaped Catalyst 1.2SB tablets, the initial ethane conversion at time=0 was 47.4 mol. % and increased to 49.6 mol. % at about time=71 hours. From time stamp ˜71 hours to the end of the run at ˜240 hours, Δ−0.09 mol. % ethane conversion was lost over this 169-hour time span, which corresponds to a 0.0005 mol. % ethane conversion absolute/hour loss.

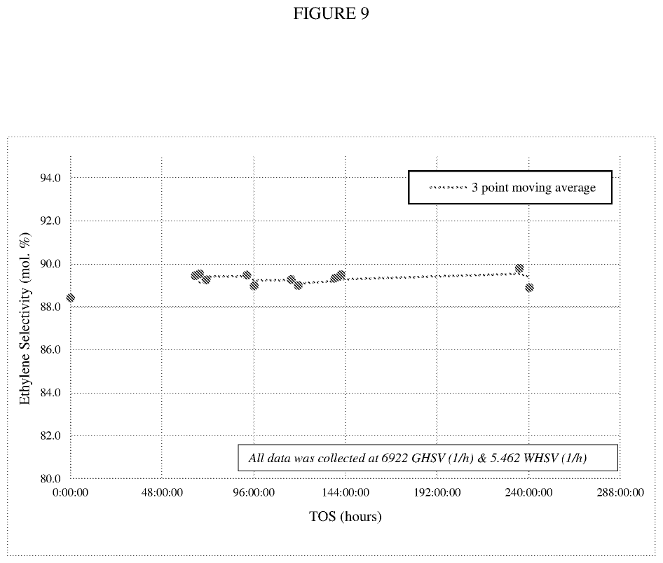

FIG. 9 shows the long-term ethylene selectivity testing results of Shaped Catalyst 1.2SB tablets. The experimental results shown in FIG. 9 were collected at 470° C. As shown in FIG. 9, the selectivity of the Shaped Catalysts 1.2 remained relatively constant over the duration of the test, at about 89 mol. % ethylene selectivity. Accordingly, these tablets display high selectivity with minimal loss over time, indicating that they are suitable for the oxidative dehydrogenation of ethane.

### Example 8: MRU Data, Shaped Catalyst 1.3SB

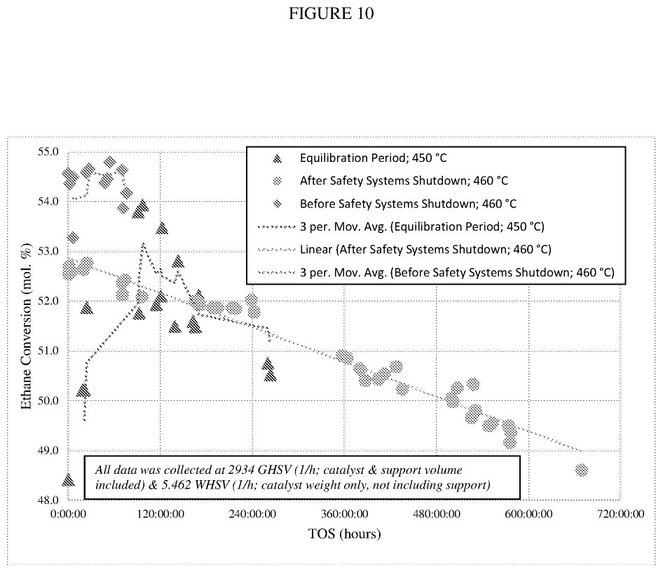

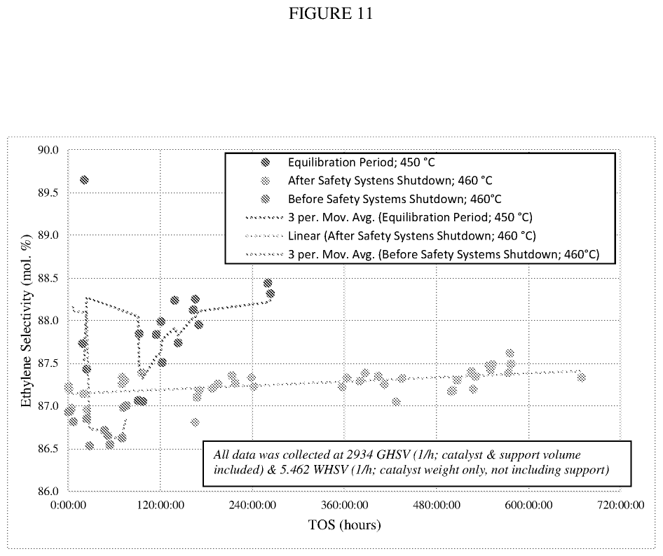

FIG. 10 and FIG. 11 show MRU data plots for MoVTeTaOx supported Shaped Catalyst 1.3SB tablets. The Shaped Catalyst 1.3SB tablets were prepared as described in Example 5.

The conversion and selectivity results of long-term testing of supported MoVTeTaOx Shaped Catalyst 1.3 tablets are presented in FIG. 10 and FIG. 11, respectively. The MRU experiments were conducted as described in the detailed description, with the following deviations. The long-term experiment of Shaped Catalyst 1.3 ran for a total duration of approximately 2176 hours at process temperatures of 450° C. (first 263 hours) and 460° C. (remaining 1913 hours). FIG. 10 shows the long-term ethane conversion testing results of the MoVTeTaOx Shaped Catalyst 1.3SB tablets. The testing ran for a total duration of approximately 2176 hours at process temperatures of 450° C. (first 263 hours) and 460° C. (remaining 1913 hours). The experiment was conducted in three stages as described herein—the equilibrium period at 450° C. (triangles), higher conversions at 460° C. before shutdown (diamonds), and higher conversions at 460° C. after shutdown (circles). All data was collected at 2934 GHSV hr⁻¹ (catalyst and support volume included) and 5.462 WHSV hr⁻¹ (catalyst weight only, not including support). The shutdowns occurred as a routine safety systems testing. However, the three data periods can be used to demonstrate the properties of the Shaped Catalyst 1.3 tablets.

From FIG. 10, it can be seen that MoVTeTaOx supported Shaped Catalyst 1.3SB demonstrated a subtle deactivation trend. However, this MRU experiment was unique in that it pushed the process conditions so that the catalyst would convert nearly all the feed oxygen content. Higher temperatures resulting in more conversion also result in more oxygen consumed to yield ethylene. Conditions where no oxygen remains can starve a catalyst of the oxygen needed to complete catalytic cycles and damage the catalyst. This was done to intentionally exaggerate the deactivation the catalyst and to determine if regeneration after deactivation is possible. As shown herein, the Shaped Catalyst 1.3SB is resistant to high conversions/low oxygen conditions without damage. Accordingly, industrial processes can run at higher conversions with less oxygen in the outlet gas product, which in turn means less downstream equipment and/or processing is required for removing residual unreacted oxygen. Further, this experiment demonstrates whether higher oxygen consuming conditions stimulate faster or slower deactivation compared to the other experiments presented herein. Without wishing to be bound by theory, it was hypothesized that aggressive oxygen consumption would deactivate the catalyst faster, similar to what is observed with MoVTeNbOx containing catalysts.

At process temperature of 450° C., MoVTeTaOx supported Shaped Catalyst 1.3SB tablets displayed an ethane conversion of 48.4 mol. % and time=0 hours and 51.9 mol. % at time ˜24 hours, shown in FIG. 10 as the “equilibration period”. From time ˜24 hours (51.9 mol. % ethane conversion) to time ˜260 hours (50.8 mol. % ethane conversion), the ethane conversion changed by Δ−1.1 mol. % abs. over this 236-hour time span, which corresponds to a 0.0085 mol. % ethane conversion absolute/hour loss.

At time stamp ˜283 hours, the MRU experiment process temperature was increased from 450° C. to 460° C., which pushed the ethane conversions higher to 54.6 mol. %. At 54.6 mol. % conversion, the amount of oxygen in the product was measured via GC to be 0.53 mol. %. It was previously established with MoVTeNbOx catalysts, running <1 mol. % oxygen in the product gas stream can lead to catalyst deactivation. In FIG. 10 this data is labeled as the “before safety systems shutdown”.

The experiment run uninterrupted for 76-hours from time ˜283 hours to time ˜359 hours until a planned shutdown occurred where the MRU unit was cooled down from 460° C. to room temperature under nitrogen gas purge. During the 76 hours uninterrupted run, the ethane conversion changed by Δ−0.4 mol. % ethane, which corresponds to a 0.0053 mol. % ethane conversion absolute/hour loss. Accordingly, these catalyst tablets are shown to be stable, with very small amounts of ethane conversion absolute/hour loss.

The MRU experiment was restarted approximately 7 days later. The experiment was resumed at a temperature of 460° C. From this point onwards, the timestamp was reset to t=0 hours for data comparison. In FIG. 10 this data is labeled as the “after safety systems shutdown”.

After the shutdown, from time ˜0 hours (52.7 mol. % ethane conversion) to time ˜674 hours (48.9 mol. % ethane conversion), the ethane conversion changed by Δ−3.8 mol. % abs., which corresponds to a 0.0056 mol. % ethane conversion absolute/hour loss.

The rate of deactivation trend before and after the safety system shutdown is essentially the same (˜0.005 mol. % ethane conversion absolute/hour) even though the amount of oxygen consumption is different. Before the system shutdown, the ethane conversion was high enough that the oxygen consumption (conversion) put the product gas at <1 mol. %. O₂ absolute, taken as grab samples injected from the live process. After the shutdown, the oxygen in the product was mainly below 1 mole % O₂, but was close to this target, in the range of 0.73-1.00 mol. % O₂. This was the case between time stamps 0-hours and ˜218 hours. After 218 hours, the amount of oxygen gas in the product gas was above 1 mol. % O₂. The similar results that the deactivation trend before and after the reactor was operated at high oxygen gas consumption indicates that the deactivation trend is not related to the amount of oxygen consumed in the reactor.

This result yields insight into the mechanism of deactivation. Without wishing to be bound by theory, redox deactivation of molybdenum-based catalysts is reported to be related to the over-reduction of molybdenum oxide active centers. Over-reduced molybdenum sites can convert to other states irreversibly changing the active phase to an inactive crystalline phase. If redox deactivation were the main mechanism of activity loss, it would be reasonable to expect that when the process conditions were pushed to higher oxygen consumption, this would have led to over-reduction of the catalyst. Over-reduction would lead to the overall activity of the catalyst bed being decreased, observable as an overall deactivation trend in the MRU data. However, if this redox deactivation mechanism was dominant, then higher oxygen consumption should accelerate the deactivation trend, which was not observed in this experiment. Accordingly, it is possible that another deactivation mechanism is at play, for example sintering deactivation. Sintering deactivation is one of the most common deactivation mechanisms for heterogeneous catalysis. As shown herein, the catalyst is stable with minimal deactivation.

FIG. 11 shows the long-term ethylene selectivity testing results of the Shaped Catalyst 1.3SB tablets. The experiment was conducted in three stages as described herein—the equilibrium period at 450° C. (triangles), higher conversions at 460° C. before shutdown (diamonds), and higher conversions at 460° C. after shutdown (circles). All data was collected at 2934 GHSV hr⁻¹ (catalyst and support volume included) and 5.462 WHSV hr⁻¹ (catalyst weight only, not including support). From FIG. 11, it can be seen that the selectivity remained relatively constant over the duration of the test ˜88 mol. % ethylene selectivity. Accordingly, these tablets display high selectivity with minimal loss over time, indicating that they are suitable for the oxidative dehydrogenation of ethane.

### Example 9: MRU Data for Shaped Catalyst 1.5SB

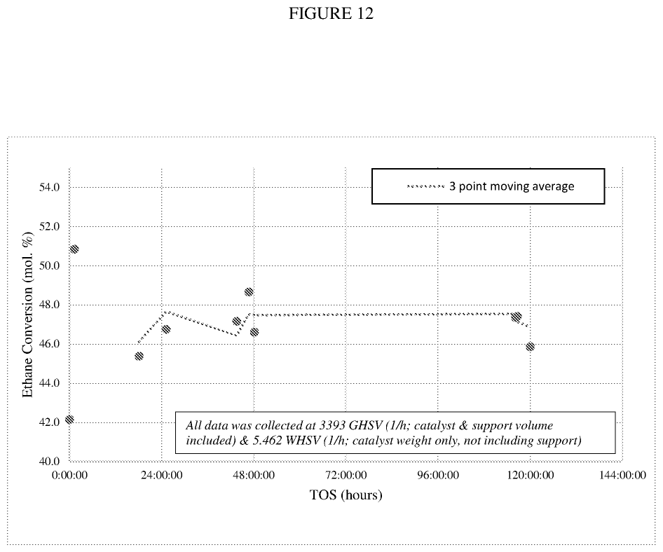

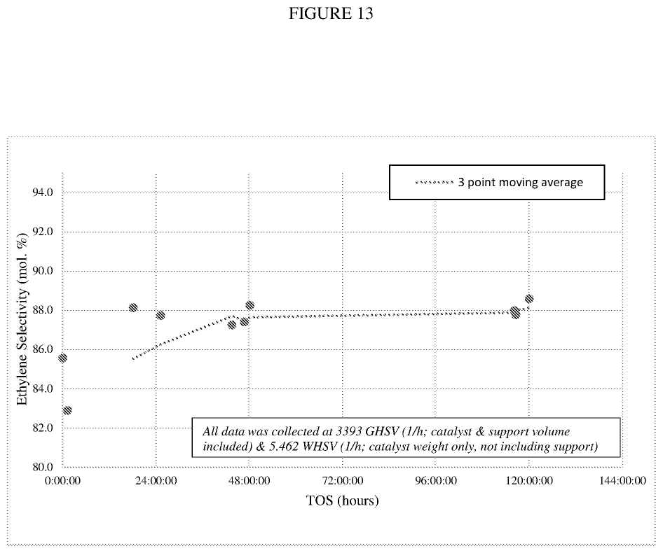

FIG. 12 and FIG. 13 show the MRU data for MoVTeTaOx supported Shaped Catalyst 1.5SB tablets. Shaped Catalysts 1.5SB were synthesized as described in Example 5. The MRU experiment was conducted as described in the detailed description, with the following parameters. The experiment was conducted for a duration of 120 hours TOS.

FIG. 12 shows the short-term ethane conversion testing results of MoVTeTaOx supported Shaped Catalyst 1.5SB tablets. The experimental results in FIG. 12 were collected at 460° C. All data was collected at 3393 GHSV hr⁻¹ (catalyst and support volume included) and 5.462 WHSV hr⁻¹ (catalyst weight only, not including support).

At time t=0, the ethane conversion was 42.1 mol. % and increased to 48.6 mol. % after ˜ 48 hours TOS. From time ˜48 hours to the end of the run at ˜120 hours, Δ−1.2 mol. % ethane conversion was lost over this 72-hour time span, which corresponds to an approximate rate of 0.0167 mol. % ethane conversion absolute/hour loss.

FIG. 13 shows the long-term ethylene selectivity testing results of MoVTeTaOx Shaped Catalyst tablet 1.5SB. The experimental results shown in FIG. 13 were collected at 460° C. All data was collected at 3393 GHSV hr⁻¹ (catalyst and support volume included) and 5.462 WHSV hr⁻¹ (catalyst weight only, not including support). As shown in FIG. 13, the selectivity remained relatively constant over the duration of the test at approximately 88 mol. % ethylene selectivity. Accordingly, these tablets display high selectivity with minimal loss over time, indicating that they are suitable for the oxidative dehydrogenation of ethane.

### Example 10: MRU Data for Shaped Catalyst 1.7SB

Shaped Catalysts 1.7SB were prepared as described in Experiment 5. The MRU process was run as described in the detailed description, with the following parameters. The long-term experiment of Shaped Catalyst 1.7SB ran for a total duration of approximately 1197 hours at a process temperature of 470° C. This experiment included intermittent air regenerations to determine if the catalyst could be regenerated proactively at set intervals. In this experiment, “air” regenerations include a gaseous mixture of nitrogen and oxygen. It was shown that the catalysts displayed improved stability with proactive air regenerations at planned intervals of times, at t=285, 643, and 980 hours. All air regenerations were conducted at 380° C. with a flow of 760 sccm with 10 vol O₂/balance N₂. All data was collected at 3323 GHSV hr⁻¹ (catalyst and support volume included) and 5.462 WHSV hr⁻¹ (catalyst weight only, not including support).

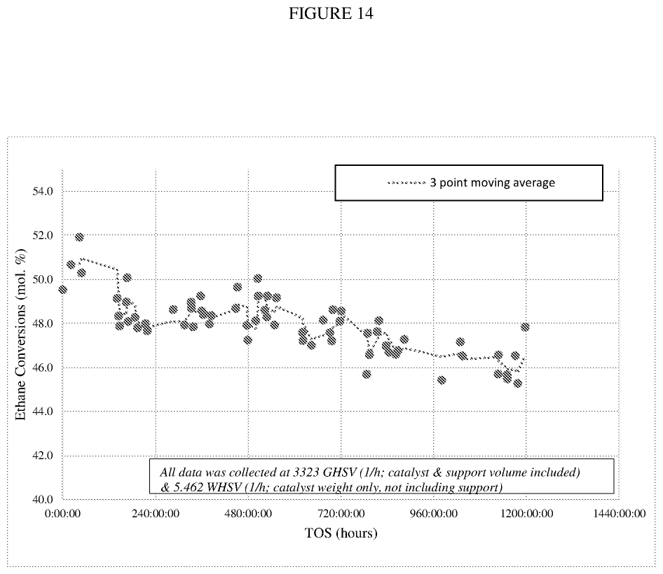

FIG. 14 shows the long-term ethane conversion testing results. The results indicated that the catalyst is workable without significant deactivation, and that regeneration is possible. Another potential means of mitigating the deactivation observed would be to change the diluent, e.g., nitrogen, to steam and/or carbon dioxide.

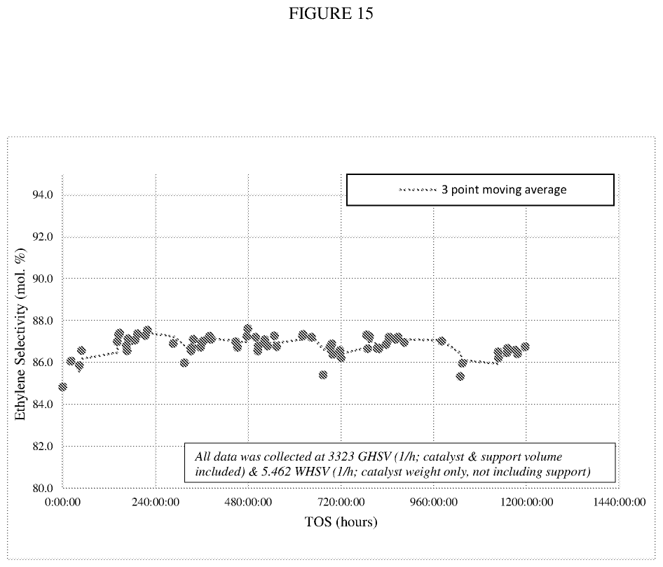

FIG. 15 shows the long-term ethylene selectivity testing results of Shaped Catalyst 1.7SB tablets. As shown in FIG. 15, selectivity remained relatively constant over the duration of the test, at approximately 87 mol. % ethylene selectivity.

The results of the first air regeneration were positive, firstly with no losses in selectivity remaining at a steady ˜87 mol. % ethylene. Secondly, the activity recovered and remained elevated for a substantial TOS duration. The ethane conversion right after air regeneration was measured to be 47.9 mol. % at experimental t≈315 hours but increased to 49.0 mol. % at t≈332 hours. Comparing 49.0 mol. % ethane conversion to the 48.0 mol. % before air regeneration corresponds to a Δ+1.0 mol. % abs. positive improvement. Advantageously, the conversion activity stayed in the 48 mol. % range until the TOS time stamp of 621 hours. The next regeneration was conducted at TOS 643 hours. Between 332 hours and 643 hours, the ethane conversion data was as follows: range=47.0-50.0 mol. %, average=48.4 mol. %, and standard deviation of data=0.78 mol. % (47.6-49.2 mol. %). Choosing an ethane conversion of 48 mol. % as the finishing point at 643 hours TOS and comparing against the initial activity of 49.5 mol. % at t=0 hours yields an ethane conversion changed of Δ−1.5 mol. % abs. over this 614-hour time span (subtracted out air regeneration time of ˜29 hours), which corresponds to a 0.0024 mol. % ethane conversion absolute/hour loss. Accordingly, the air regenerations can successfully regenerate the catalysts and can be used to maintain constant conversion for the processes under normal operating conditions.

### Example 11: MRU Data for Shaped Catalysts 1.8SB

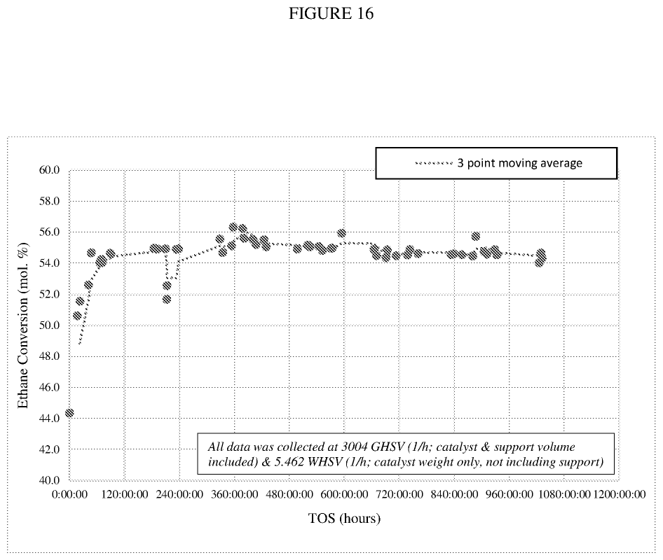

Shaped Catalysts 1.8SB were prepared as described in Experiment 5. The MRU process was run as described in the detailed description, with the following parameters. The long-term experiment of Shaped Catalyst 1.8 ran for a total duration of approximately 1032 hours at a process temperature of 420° C. FIG. 16 shows the long-term ethane conversion of the Shaped Catalysts 1.8SB. As shown in FIG. 16, the MoVTeNbOx, supported Shaped Catalyst 1.8 Tablets demonstrated exceptional stability.

At process temperature of 420° C., MoVTeNbOx Shaped Catalyst 1.8 tablets ethane conversions started at 44.4 mol. % and time=0 hours and increased to 54.7 mol. % at time≈47 hours. From time ˜47 hours (54.7 mol. % ethane conversion) to time ˜357 hours (56.3 mol. % ethane conversion), the ethane conversion changed by Δ+1.6 mol. % abs. over this 310-hour time span, which corresponds to 0.0052 mol. %_{ethane conversion absolute}/hour gain. Overall, from time=0 hours to time≈357 hours, the ethane conversion changed by Δ+11.9 mol. % abs., which is a desirable result. The entirety of the long-term testing of supported MoVTeNbOx Shaped Catalyst 1.8 tablets was conducted with no air regenerations. There was a shutdown at time≈212 hours, during which the feed gas cut off and a nitrogen purge was initiated while the process cooled down from 420° C. However, this nitrogen purge period was brief (<1 hour) and the resulting activity loss was short-lived and catalyst behavior fully recovered by time≈233 hours and even improved up to time≈357 hours. From the time stamp of TOS 357 hours, a subtle deactivation trend started. At time stamp TOS 1032 hours, the ethane conversion was measured to be 54.4 mol. % ethane conversion (an average of the last 4 data points measured). The ˜1000-hour deactivation trend, relative to ethane conversions collected at time≈47 hours (54.7 mol. % ethane conversion), is Δ−0.3 mol. % abs. based on the 4-point average of 54.4 mol. % result obtained at the end of the experiment (t≈1032 hours). An ethane conversion loss of Δ−0.3 mol. % abs. corresponds to 0.0003 mol. % ethane conversion absolute/hour loss over the 985-hour experimental period.

The increase in ethane conversion was an unexpected result, and demonstrates that the fumed silica support has a synergistic effect with shaped catalysts that include both MoVTeNbOx and MoVTeTaOx catalyst active phases.

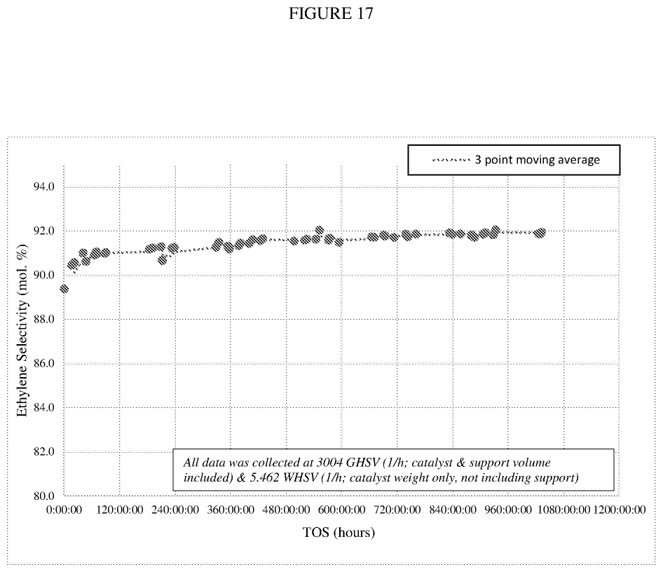

FIG. 17 shows the ethylene selectivity of the Shaped Catalyst 1.8SB tablets. As shown in FIG. 17, the selectivity actually increased over the entire duration of the test, from ˜90 mol. % to 92 mol. % ethylene selectivity. Again, this is an unexpected but desirable result. Accordingly, these tablets display high selectivity with minimal loss over time, indicating that they are suitable for the oxidative dehydrogenation of ethane.

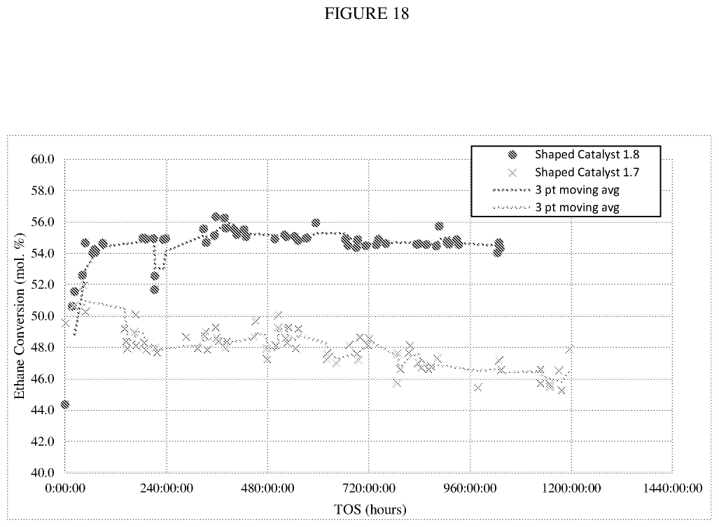

FIG. 18 shows the long-term ethylene selectivity testing of Shaped Catalyst 1.7SB and Shaped Catalyst 1.8SB, overlaid into a single plot. Table 7 summarizes the catalyst performance data for these shaped catalysts. Both shaped catalyst show little to no deactivation trend. The slight increase in deactivation trend of the Shaped Catalyst 1.7SB compared to Shaped Catalyst 1.8SB may be due to the higher temperatures used to analyze the Shaped Catalyst 1.7SB.

TABLE 7
Summary of Catalyst Performance Data
for Shaped Catalysts 1.7SB and 1.8SB

Catalysts

| | Shaped | Shaped |
|---|---|---|
| | Catalyst | Catalyst |
| | 1.7SB | 1.8SB |
| Mean Axial Crush Strength (N) | 477 | 117 |
| Crush Strength SD (N) | 66 | 60 |
| Mean Radial Crush Strength (N) | 51 | 28 |
| Crush Strength SD (N) | 15 | 5 |
| MRU LT Run Temp (° C.) | 470 | 420 |
| MRU LT run GHSV for Total | 3323 | 3004 |
| Supported Catalyst (h−1) | | |
| Initial Activity (mol. % C2H6) | ~50 | ~51 |
| Initial Activity (mol. % C2H6) | 87 | 90 |
| MRU TOS (hours) | 1197 | 887 |
| # of Regenerations | 3 | 0 |
| Current TOS (mol. % C2H6) | ~46 | ~55 |
| ~Δ in mol. % C2H6 Conversion | ~−4 | ~+4 |
| Absolute after TOS | | |
| Current TOS mol. % C2H4 Selectivity | ~87 | ~92 |
| ~Δ in mol. % C2H6 Conversion | 0 | ~+2 |
| Absolute after TOS | | |
| HGGC Average mol. % C2H3O2H | 0.02 | 0.04 |
| Selectivity | | |

The following units of measure have been mentioned in this disclosure:

| Unit of Measure | Full form |
|---|---|
| g | Gram |
| ° C. | Degrees Celsius |
| mL | Milliliter |
| μm | Millimeter |
| mm³ | Cubic millimeter |
| g/cm³ | Grams per cubic centimeter |
| μm | Micrometer |
| sccm | Standard cubic centimeter per minute |
| psig | Pounds per square inch gauge |
| MPa | Megapascal |
| wt. % | Weight percent |
| mol. % | Mole percent |
| N | Newtons |
| hr | Hour |
| hr⁻¹ | Per hour |

The term “about” as used in this disclosure can allow for a degree of variability in a value or range, for example, within 10%, within 5%, or within 1% of a stated value or of a stated limit of a range.

The term “substantially” as used in this disclosure refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “solvent” as used in this disclosure refers to a liquid that can dissolve a solid, another liquid, or a gas to form a solution. Non-limiting examples of solvents are silicones, organic compounds, water, alcohols, ionic liquids, and supercritical fluids.

The term “room temperature” as used in this disclosure refers to a temperature of about 15 degrees Celsius (° C.) to about 28° C.

As used in this disclosure, “weight percent” (wt. %) can be considered a mass fraction or a mass ratio of a substance to the total mixture or composition. Weight percent can be a weight-to-weight ratio or mass-to-mass ratio, unless indicated otherwise.

A number of implementations of the disclosure have been described. Nevertheless, it will be understood that various modifications may be made without departing from the spirit and scope of the disclosure.

1. A composition comprising:
a catalyst active phase, wherein the catalyst active phase comprises a MoVTeNbOx catalyst; and
a support phase, wherein the support phase comprises fumed silica, wherein the catalyst active phase and support phase form a heterogeneous mixture.

2. The composition of claim 1, further comprising a binder, wherein the binder comprises a polymer organic acid, amine, or alcohol.

3. The composition of claim 2, wherein the binder comprises polyvinyl alcohol or polyethylene glycol, or both.

4. The composition of claim 3, wherein the binder comprises 60-80 wt. % polyvinyl alcohol and 20-40 wt. % polyethylene glycol.

5. The composition of claim 1, further comprising an inorganic binder, wherein the inorganic binder comprises talc or calcium carbonate.

6. The composition of claim 1, further comprising sintering the composition to form a ceramic.

7. The composition of claim 1, wherein the composition further comprises a plasticizer, wherein the plasticizer comprises polyethylene glycol.

8. The composition of claim 1, wherein the composition further comprises a lubricant.

9. The composition of claim 8, wherein the lubricant is 1-2 wt. % of the composition, and wherein the lubricant comprises graphite.

10. The composition of claim 1, wherein the fumed silica is 5-95 wt. % of the composition.

11. A pressed tablet comprising the composition of claim 1, wherein the pressed tablet has a density of 1.0-1.5 g/cm³.

12. A sintered ceramic catalyst comprising the composition of claim 1.

13. A method of making a shaped catalyst, the method comprising:
combining a catalyst active phase, a support phase, and a binder in an aqueous solvent to form an aqueous solution, wherein the catalyst active phase comprises MoVTeNbOx and the support phase comprises fumed silica;
heating the aqueous solution to form a thick paste;
drying the thick paste to evaporate the aqueous solvent to form a solid powder;
grinding the solid powder to form granules; and
pressing the granules to form a tablet.

14. The method of claim 13, wherein grinding the solid powder to form granules comprises grinding the solid powder to form granules wherein the granules are between 105 μm and 710 μm in diameter.

15. The method of claim 13, further comprising:
debinding the tablet, wherein debinding the tablet comprises heating the tablet to remove the binder; and
sintering the tablet, wherein sintering the tablet comprises heating the tablet to coalesce the tablet into a densified solid.

16. The method of claim 15, wherein debinding the tablet comprises heating the tablet to 200° C.-400° C. at a rate of 1-2° C. per minute.

17. The method of claim 15, wherein sintering the tablet comprises heating the tablet to 400° C.-600° C. at a rate of up to 5° C. per minute.

18. A method of making a shaped catalyst, the method comprising:
combining a catalyst active phase, a support phase, and a binder in an aqueous solvent to form an aqueous solution, wherein the catalyst active phase comprises MoVTeNbOx and the support phase comprises fumed silica;
heating the aqueous solution to form a thick paste;
drying the thick paste to evaporate the aqueous solvent to form a solid powder;
grinding the solid powder to form granules;
pressing the granules to form a tablet;
debinding the tablet, wherein debinding the tablet comprises heating the tablet to remove the binder; and
sintering the tablet, wherein sintering the tablet comprises heating the tablet to coalesce the tablet into a densified solid.

19. A method of oxidative dehydrogenation of ethane, the method comprising:
flowing a feed gas through a microreactor unit, wherein the feed gas comprises ethane, oxygen, and nitrogen;
pressuring the feed gas in the microreactor;
contacting the feed gas with a shaped catalyst, the shaped catalyst comprising:
a catalyst active phase, wherein the catalyst active phase comprises a MoVTeNbOx catalyst; and
a support phase, wherein the support phase comprises fumed silica, wherein the catalyst active phase and support phase form a heterogeneous mixture heating the feed gas to 450° C.-470° C.; and
recovering ethylene gas.

20. The method of claim 19, wherein the feed gas comprises a 1:0.5 mole ratio of ethane to oxygen.
